(19) United States
(12) Patent Application Publication
(10) Pub. No.: US 2016/0320384 A1
(43) Pub. Date: Nov. 3, 2016

(54) METHODS FOR ASSAYING IMMUNOLOGICAL COMPETENCE

(71) Applicant: YEDA RESEARCH AND DEVELOPMENT CO. LTD., Rehovot (IL)

(72) Inventors: Irun R. Cohen, Rehovot (IL); Eytan Domany, Rehovot (IL); Noam Shental, Rehovot (IL); Ittai Fattal, Rehovot (IL)

(73) Assignee: YEDA RESEARCH AND DEVELOPMENT CO. LTD., Rehovot (IL)

(21) Appl. No.: 15/107,483

(22) PCT Filed: Dec. 31, 2014
(86) PCT No.: PCT/IL2014/051141

§ 371 Date: Jun. 23, 2016

Related U.S. Application Data
(60) Provisional application No. 61/922,112, filed on Dec. 31, 2013.

(51) Int. Cl. G01N 33/564; C07K 14/705; A61K 38/13; C12Q 1/68; G01N 33/68

(52) U.S. Cl. CPC G01N 33/564; C12Q 1/6804; G01N 33/6854; A61K 38/13; C07K 14/70521; G01N 2800/245; G01N 2800/52; C07K 2319/30; A61K 38/00

(57) ABSTRACT

A method of assaying or monitoring the determining immunological competence of a subject, particularly for determining immunological competence in a subject, including but not limited to a transplant recipient, is provided. The method comprises measuring the levels of antibodies in a sample obtained from a subject to poly-guanine oligonucleotides.

### FIELD OF THE INVENTION

The present invention relates to a method of assaying and monitoring the immune response and particularly determining immunological competence or lack thereof in subjects. The methods of the invention comprise measuring the level of antibodies in a sample obtained from a subject to oligonucleotide sequences such as poly-guanine oligonucleotides.

### BACKGROUND OF THE INVENTION

Patients who receive a solid organ transplant must take immunosuppressive therapy to prevent rejection. Contemporary immunosuppressive protocols call for continuous therapy for the life-span of the transplanted organ. Potentially life-long anti-rejection therapy has many adverse consequences including increased rates of infections and cancers, cardiovascular risk factors and bone diseases. Therefore, individualized or minimized immunosuppression is a major clinical goal, saving the highest levels of immunosuppressive therapy for those patients at higher risk of rejection and graft loss.

Methods for monitoring the immune response and predicting clinical outcomes for patients on immunosuppressive drugs (such as transplant patients) are disclosed in U.S. Pat. No. 7,476,514. The methods are based on the measurement of an intra-cellular metabolic marker in lymphocytes (such as ATP) as an indicator of a patient's immune response. Additional patent applications relating to evaluating immunosuppression based on NFkB levels include U.S. Application Nos. 2011/0312016 and 2013/0183686.

Recent developments using antigen microarray devices and informatics analyses made it possible to profile microliter amounts of serum for quantitatively binding of antibodies to hundreds of different molecules (Merbl Y, et al. J Clin Invest 2007; 117(3):712-8; Quintana F J, et al. Lupus 2006; 15:428-30; Fattal I, et al. Immunology 2010; 130(3): 337-43).

Antibodies to DNA are important markers of various autoimmune diseases and can be pathogenic; however, their generation is not understood. Herkel J., Cohen I R. et al. (Eur J Immunol. 2004 December; 34(12):3623-32) reported that a monoclonal anti-DNA antibody raised as an anti-idiotype to an antibody to a DNA-binding domain of p53 could, like p53, bind to a 20-mer poly-G homo-oligonucleotide but not to a poly-T 20-mer.

International Patent Application Publication No. WO 11/099012, to some the present inventors, relates to methods and kits for diagnosing systemic lupus erythematosus (SLE) in a subject, using a specific antibody profile. The '012 publication discloses patients having, inter alia, increased IgG reactivity to Epstein-Barr Virus (EBV). Additional patents and patent applications disclosing diagnosis of autoimmune diseases using a specific antibody profile include WO 10/055510, WO 12/052994, US 2005/0260770 and U.S. Pat. No. 8,010,298. Further, US Patent Application Publication No. 2012/0122720 relates to recognizing the development of cardiovascular disease, e.g., acute myocardial infarction process in an individual. International Patent Application Publication No. WO 2014/091490, of some the present inventors, relates to methods for diagnosing SLE or scleroderma by using specific antibody profiles against an array of antigens derived from the Epstein-Barr Virus (EBV).

Currently, there is an ongoing need for a reliable, non-invasive test for detecting immunological competence is subjects such as transplant patients. The present invention meets this need.

### SUMMARY OF THE INVENTION

The present invention provides a method of detecting immunological competence or lack thereof in a subject, including but not limited to a transplant recipient and/or a subject receiving at least one immunosuppressive drug. In some embodiments, the methods of the invention comprise determining the levels of antibodies in a sample obtained from a subject to poly-oligonucleotides comprising or consisting poly-guanine oligonucleotides.

The present invention is based, in part, on the surprising finding that healthy subjects as well as patients having autoimmune diseases manifest relatively high amounts of antibody binding to poly-guanine oligonucleotide. Unexpectedly, subjects treated with an immunosuppression drug showed reduced autoantibody reactivity to guanine oligonucleotides of various lengths (14-40 contiguous guanine nucleotides). Thus, quantification of antibodies to particular oligonucleotide sequences (e.g., guanine oligonucleotide sequences) may be used in various embodiments to determine the immunological competence or state of immunosuppression in an individual.

Thus, the present invention provides methods and means for assaying the level of one or more markers indicative of immunological competence and/or immunosuppression in an individual. In some embodiments, the methods are useful for determining and/or predicting whether an organ recipient is at risk of rejection of the transplanted organ. In additional embodiments, the methods are useful for determining insufficient or excessive immunosuppression in a subject undergoing immunosuppression therapy.

In particular embodiments, the present invention provides highly specific, reliable, accurate and discriminatory assays. The present invention further provides antigen probe arrays for practicing such assays, and antigen probe sets for generating such arrays.

According to a first aspect, the present invention provides a method of determining a level of immunological competence of a subject, the method comprising obtaining a sample from the subject; assaying the sample for the presence of antibodies to at least one oligonucleotide antigen comprising at least 10 contiguous guanine nucleotides, thereby determining the reactivity of the antibodies in the sample to the at least one oligonucleotide antigen; and comparing the antibody reactivity to the reactivity of a control subject or a reference control value; wherein an equal or higher reactivity of the antibodies in the sample obtained from the subject compared to the reactivity of the control subject is an indication that the subject has a competent immunological system, and wherein a significantly lower reactivity of the antibodies in the sample obtained from the subject compared to control is an indication that the subject has an incompetent immunological system.

In certain embodiments, the method of determining a level of immunological competence of a subject or lack thereof comprises assaying for the presence of antibodies in a sample obtained from the subject to at least one antigen comprising an oligonucleotide sequence comprising at least 10 contiguous guanine nucleotides, thereby determining the reactivity of the antibodies in the sample to the at least one antigen and comparing the antibody reactivity to the reactivity of a control subject or a reference control value. In some embodiments, an equal or higher reactivity of the antibodies in the sample obtained from the subject compared to the reactivity of the control subjects is an indication that the subject has a competent immunological system. According to other embodiments, a significantly lower reactivity of the antibodies in the sample obtained from the subject compared to the reactivity of the control subjects is an indication that the subject has an incompetent immunological system. According to particular embodiments, a significantly lower reactivity of the antibodies in the sample obtained from the subject compared to the reactivity of the control subjects is an indication that the subject has a suppressed immunological system.

According to a related aspect, the present invention provides a method of determining immunological competence or immunological incompetence of a subject, the method comprising assaying for the presence of antibodies in a sample obtained from the subject to at least one oligonucleotide antigen comprising at least 10 contiguous guanine nucleotides, thereby determining the reactivity of the antibodies in the sample to the at least one oligonucleotide antigen, and comparing the antibody reactivity to the reactivity of a control subject or a reference control value, wherein an equal or higher reactivity of the antibodies in the sample obtained from the subject compared to the reactivity of the control subject is an indication that the subject has a competent immunological system, and wherein a significantly lower reactivity of the antibodies in the sample obtained from the subject compared to control is an indication that the subject has an incompetent immunological system.

According to a further related aspect, the present invention provides a method of determining immunological competence or suppression of a subject, the method comprising obtaining a sample from the subject, assaying the sample for the presence of antibodies to at least one oligonucleotide antigen comprising at least 10 contiguous guanine nucleotides, thereby determining the reactivity of the antibodies in the sample to the at least one oligonucleotide antigen, and comparing the antibody reactivity to the reactivity of a control subject or a reference control value, wherein an equal or higher reactivity of the antibodies in the sample obtained from the subject compared to the reactivity of the control subject is an indication that the subject has a competent immunological system, and wherein a significantly lower reactivity of the antibodies in the sample obtained from the subject compared to control is an indication that the subject has an incompetent immunological system.

According to some embodiments, the subject is a transplant patient or an organ recipient or is being evaluated as an organ recipient candidate (i.e., a transplant candidate). According to additional embodiments, the subject is or was receiving at least one immunosuppressive drug or an immunosuppressive treatment. According to some embodiments, the at least one immunosuppressive treatment is irradiation. According to some embodiments, the at least one immunosuppressive drug is selected from the group consisting of: corticosteroids, calcineurin inhibitors (CNIs) including but not limited to cyclosporine or tacrolimus, T-cell costimulatory blocker such as belatacept, purine metabolism inhibitors including but not limited to azathioprine or mycophenolate mofetil (MMF), mammalian Target Of Rapamycin (mTOR) inhibitors including but not limited to sirolimus or everolimus, immunosuppressive Immunoglobulins (Ig) including but not limited to antilymphocyte globulin (ALG) and antithymocyte globulin (ATG), monoclonal antibodies (mAbs) including but not limited to OKT3 or anti-interleukin-2 (IL-2) receptor monoclonal antibodies. In certain embodiments, the at least one immunosuppressive drug is a T-cell costimulatory blocker, such as belatacept. In certain embodiment, the at least one immunosuppressive drug is a calcineurin inhibitor, such as cyclosporine A.

According to another embodiment, the subject receiving at least one immunosuppressive drug has an autoimmune disease. Non-limiting examples of autoimmune diseases are lupus, multiple sclerosis, rheumatoid arthritis or Crohn's disease. According to another embodiment, the subject is an immunocompromised subject, such as a subject undergoing chemotherapy treatment, a subject afflicted with cancer including but not limited to leukemia, lymphoma, multiple myeloma, or a subject afflicted with a chronic infections such as acquired immunodeficiency syndrome (AIDS). Each possibility represents a separate embodiment of the present invention.

According to some embodiments, the reactivity of antibodies is selected from IgG reactivities and IgM reactivities. In one embodiment, the reactivity of antibodies is IgG reactivities. In another embodiment, the reactivity of antibodies is IgM reactivities.

In certain embodiments, the method of the present invention is indicative to a change of at least 19%, at least 28%, at least 30%, at least 33%, at least 37%, at least 41%, at least 42%, at least 47% or at least 55% in the level of the immunological competence of a subject, when comparing the antibody reactivity to the reactivity of a control subject or a reference control value. In certain embodiments, the method of the present invention is indicative to a change of at least 28%, at least 37%, at least 41%, at least 55% in the level of the immunological competence of a subject, when comparing the IgM antibody reactivity to the reactivity of a control subject or a reference control value. In certain embodiments, the method of the present invention is indicative to a change of at least 19%, at least 30%, at least 33%, at least 42 or at least 47% in the level of the immunological competence of a subject, when comparing the IgG antibody reactivity to the reactivity of a control subject or a reference control value. Each possibility represents a separate embodiment of the invention.

According to another embodiment, the at least one oligonucleotide antigen is selected from the group consisting of G14 having the nucleotide sequence as set forth in SEQ ID NO: 41, G20 having the nucleotide sequence as set forth in SEQ ID NO: 43, G40 having the nucleotide sequence as set forth in SEQ ID NO: 66, G17 having the nucleotide sequence as set forth in SEQ ID NO: 36, and G30 having the nucleotide sequence as set forth in SEQ ID NO: 65. In certain embodiments, the at least one antigen is G14 having the nucleotide sequence as set forth in SEQ ID NO: 41. In certain embodiments, the at least one antigen is G20 having the nucleotide sequence as set forth in SEQ ID NO: 43.

In one embodiment, the at least one oligonucleotide antigen is G14 having the nucleotide sequence as set forth in SEQ ID NO: 41. In one embodiment, the at least one oligonucleotide antigen is G20 having the nucleotide sequence as set forth in SEQ ID NO: 43. In one embodiment, the at least one oligonucleotide antigen comprises the nucleotide sequence as set forth in SEQ ID NO: 43 (GGGGGGGGGGGGGGGGGGGG). In an exemplary embodiment, the at least one oligonucleotide antigen consists of the nucleotide sequence as set forth in SEQ ID NO: 43. In one embodiment, the at least one oligonucleotide antigen comprises the nucleotide sequence as set forth in SEQ ID NO: 38 (TGGGGGGGGGGGGGGGG). In another embodiment, the oligonucleotide sequence further comprises at least one thymine at the 5′ terminus of the oligonucleotide sequence.

In certain embodiments, the oligonucleotide antigen consists of an oligonucleotide sequence set forth in SEQ ID NO: 10, 11, 12, 18, 19, 20, 24, 27, 31, 32, 36, 38, 41, 43, 47, 60, 62, 65 or 66. Each possibility represents a separate embodiment of the present invention. In certain embodiments, the oligonucleotide antigen consists of an oligonucleotide sequence selected from the group consisting of the oligonucleotide sequence set forth in SEQ ID NOs: 19, 24, 27, 31, 32, 36, 38, 41, 43, 47, 60, 62, 65 and 66. In certain embodiments, the oligonucleotide sequence does not comprise one or more thymine at the 3′ terminus of the oligonucleotide sequence. Each possibility represents a separate embodiment of the present invention.

According to some embodiments, the method comprises determining the reactivity of antibodies in the sample obtained from the subject to at least two different oligonucleotide antigens. According to some embodiments, the at least two different oligonucleotide antigens are selected from the group consisting of G14 having the nucleotide sequence as set forth in SEQ ID NO: 41, G20 having the nucleotide sequence as set forth in SEQ ID NO: 43, G40 having the nucleotide sequence as set forth in SEQ ID NO: 66, G17 having the nucleotide sequence as set forth in SEQ ID NO: 36, and G30 having the nucleotide sequence as set forth in SEQ ID NO: 65. In some embodiments, the method comprises determining the reactivity of antibodies in the sample obtained from the subject to G20 antigen (SEQ ID NO: 43) and at least one additional antigen selected from SEQ ID NO: 36, 41, 65 and 66. According to additional embodiments, the method comprises determining the reactivity of antibodies in the sample obtained from the subject to at least three, at least four, at least five or more antigens.

According to another embodiment, the control is selected from the group consisting of a sample from at least one control individual, a panel of control samples from a set of control individuals, a stored set of data from a set of control individuals, and a reference control value from a set of control individuals. In one embodiment, the control reflects the reactivity level of a subject or a set of subjects having a competent immunological system.

According to additional embodiments of the methods of the invention, the sample obtained from the subject is a biological fluid. According to some embodiments, the sample is selected from the group consisting of plasma, serum, blood, cerebrospinal fluid, synovial fluid, sputum, urine, saliva, tears, lymph specimen, or any other biological fluid known in the art. Each possibility represents a separate embodiment of the invention. According to certain embodiments, the sample obtained from the subject is selected from the group consisting of serum, plasma and blood. According to one embodiment, the sample is a serum sample.

According to some embodiments, the at least one oligonucleotide antigen is used in the form of an antigen probe set. According to some embodiments, the reactivity of antibodies to at least one oligonucleotide antigen is determined using an antigen chip or antigen array.

According to certain embodiments, the method described above further comprises the step of determining that a subject who has a competent immunological system is not amenable for organ transplantation. According to certain embodiments, the method described above further comprises the step of determining that a subject who has a suppressed or an incompetent immunological system is amenable for organ transplantation. According to certain embodiments, the method described above further comprises the step of determining that a subject who has a competent immunological system and receives immunosuppressive treatment or an immunosuppressive drug is amenable for higher dosages of the immunosuppressive treatment or the immunosuppressive drug than previously administered. According to certain embodiments, the method described above further comprises the step of determining that a subject who has a suppressed or an incompetent immunological system and receiving immunosuppressive treatment or an immunosuppressive drug is amenable for lower dosages of the immunosuppressive treatment or the immunosuppressive drug than previously administered.

According to another aspect, the present invention provides an antigen probe set comprising a plurality of different oligonucleotide antigens, each oligonucleotide antigen comprising at least 10 contiguous guanine nucleotides. Different oligonucleotide antigens are considered as having non-identical sequences and/or non-identical lengths.

According to certain embodiments, the antigen probe described above comprises a plurality of different oligonucleotide antigens selected from the group consisting of G14 having the nucleotide sequence as set forth in SEQ ID NO: 41, G20 having the nucleotide sequence as set forth in SEQ ID NO: 43, G40 having the nucleotide sequence as set forth in SEQ ID NO: 66, G17 having the nucleotide sequence as set forth in SEQ ID NO: 36, and G30 having the nucleotide sequence as set forth in SEQ ID NO: 65.

According to another aspect, the present invention provides an article of manufacture comprising the antigen probe set described above. In some embodiments, the article of manufacture is useful for determining immunological competence of a subject or lack thereof.

In certain embodiments, the article of manufacture is in the form of an antigen probe array or in the form of an antigen chip or in the form of a dipstick or in the form of a lateral flow test. In certain embodiments, the article of manufacture is in the form of a kit.

In certain embodiments, the kit further comprises means for determining the reactivity of antibodies in a sample to at least one oligonucleotide antigen of the antigen probe chip or array.

In certain embodiments, the article of manufacture further comprises means for performing the method described above, or instructions for use.

According to another aspect, there is provided the use of the at least one oligonucleotide antigen of the invention for the preparation of an antigen probe set, an antigen probe array, an antigen chip or a kit, optionally for determining immunological competence or immunological incompetence of a subject.

Other objects, features and advantages of the present invention will become clear from the following description and drawings.

### BRIEF DESCRIPTION OF THE DRAWINGS

FIG. 1 depicts individual IgM and IgG reactivities to A20 (SEQ ID NO: 22), C20 (SEQ ID NO: 15), G20 (SEQ ID NO: 43) and T20 (SEQ ID NO: 8) in sera from healthy subjects (squares), pemphigus vulgaris (PV) patients (stars), scleroderma (SSc) patients (diamonds), and SLE patients (circles). Subjects were ordered from left to right according to their reactivity to dsDNA.

FIG. 2 shows IgG reactivity to G20 (SEQ ID NO: 43) compared to all other oligonucleotides in healthy persons, SSc patients, SLE patients who are negative or positive for dsDNA. Y axis—reactivities for G20 (SEQ ID NO: 43), X axis—reactivities to oligonucleotides. The numbers that appear on both axes are ×10,000.

FIG. 3 shows mean IgM and IgG binding to poly-G and poly-T oligonucleotides as a function of chain length.

FIG. 4 depicts IgM and IgG reactivities to G17 (SEQ ID NO: 36) oligonucleotide compared to T1G16 (SEQ ID NO: 38) and G16T1 (SEQ ID NO: 18).

FIGS. 5A-B depict IgM and IgG reactivities to modified T17 oligonucleotides G1T16 (SEQ ID NO: 42) and T16G1 (SEQ ID NO: 7) (5A) and G2T16 (SEQ ID NO: 14) and T16G2 (SEQ ID NO: 28) (5B) compared to T17 (SEQ ID NO: 2) reactivities.

FIG. 6 shows IgG and IgM binding to (CG)10 (SEQ ID NO: 25) in healthy subjects, and SSc and SLE patients.

FIGS. 7A-B show the effect of immunosuppressive treatment with belatacept or CsA on the intensity (%) of G14 IgM (7A) or IgG (7B) at baseline—before immunosuppressive treatment—and 24 weeks after initiation of immunosuppressive treatment as a result of transplantation (Tx). The ratio between 24 weeks after transplantation and baseline intensities of G14 intensity is presented. The left panel presents the ratio of each of the samples obtained from subjects before and after treatment with belatacept and the right panel presents the effects on the ratio of each sample from subjects treated with CsA. The X axis presents the patients' designation number. Black circles represent intensity at baseline and white circles represent the intensity after 24 weeks of immunosuppressive treatment.

FIGS. 8A-B show the effect of immunosuppressive treatment with belatacept or CsA on the intensity (%) of G17 IgM (8A) or IgG (8B) at baseline—before immunosuppressive treatment—and 24 weeks after initiation of immunosuppressive treatment as a result of transplantation (Tx). The ratio between 24 weeks after transplantation and baseline intensities of G14 intensity is presented. The left panel presents the ratio of each of the samples obtained from subjects before and after treatment with belatacept and the right panel presents the effects on the ratio of each sample from subjects treated with CsA. The X axis presents the patients' designation. Black circles represent intensity at baseline and white circles represent the intensity after 24 weeks of immunosuppressive treatment.

FIGS. 9A-B show the effect of immunosuppressive treatment with belatacept or CsA on the intensity (%) of G20 IgM (9A) or IgG (9B) at baseline—before immunosuppressive treatment—and 24 weeks after initiation of immunosuppressive treatment as a result of transplantation (Tx). The ratio between 24 weeks after transplantation and baseline intensities of G14 intensity is presented. The left panel presents the ratio of each of the samples obtained from subjects before and after treatment with belatacept and the right panel presents the effects on the ratio of each sample from subjects treated with CsA. The X axis presents the patients' designation. Black circles represent intensity at baseline and white circles represent the intensity after 24 weeks of immunosuppressive treatment.

FIGS. 10A-B show the effect of immunosuppressive treatment with belatacept or CsA on the intensity (%) of G30 IgM (10A) or IgG (10B) at baseline—before immunosuppressive treatment—and 24 weeks after initiation of immunosuppressive treatment as a result of transplantation (Tx). The ratio between 24 weeks after transplantation and baseline intensities of G14 intensity is presented. The left panel presents the ratio of each of the samples obtained from subjects before and after treatment with belatacept and the right panel presents the effects on the ratio of each sample from subjects treated with CsA. The X axis presents the patients' designation. Black circles represent intensity at baseline and white circles represent the intensity after 24 weeks of immunosuppressive treatment.

FIGS. 11A-B show the effect of immunosuppressive treatment with belatacept or CsA on the intensity (%) of G40 IgM (11A) or IgG (11B) at baseline—before immunosuppressive treatment—and 24 weeks after initiation of immunosuppressive treatment as a result of transplantation (Tx). The ratio between 24 weeks after transplantation and baseline intensities of G14 intensity is presented. The left panel presents the ratio of each of the samples obtained from subjects before and after treatment with belatacept and the right panel presents the effects on the ratio of each sample from subjects treated with CsA. The X axis presents the patients' designation. Black circles represent intensity at baseline and white circles represent the intensity after 24 weeks of immunosuppressive treatment.

FIGS. 12A-B show the effect of immunosuppressive treatment with belatacept or CsA on the intensity (%) of combined IgG (12B) and IgM (12A) poly-G oligos at baseline—before immunosuppressive treatment—and 24 weeks after initiation of immunosuppressive treatment as a result of transplantation (Tx). The ratio between 24 weeks after transplantation and baseline intensities of poly-G oligos intensity is presented. The upper panel (12A) presents IgM median intensity and the lower panel (12B) presents the IgG median intensity.

### DETAILED DESCRIPTION OF THE INVENTION

The present invention provides methods and means for assaying and determining immunological competence and/or immunosuppression in an individual. In some embodiments, the methods are useful for determining and/or predicting whether an organ recipient is at risk of rejection of the transplanted organ. In particular embodiments, the present invention provides highly specific, reliable, accurate and discriminatory assays. The present invention further provides antigen probe arrays for practicing such methods, and antigen probe sets for generating such arrays.

The present invention is based, in part, on the surprising finding that healthy subjects as well as patients having autoimmune diseases manifest relatively high amounts of IgG and IgM antibodies capable of binding to a 20-mer guanine oligonucleotide as opposed to very low or no reactivities to 20-mer adenine/cytosine/thymine oligonucleotides. Furthermore, an unexpected reduction in poly-G autoantibody reactivity, including antibody binding to G14 (SEQ ID NO: 41), G17 (SEQ ID NO: 36), G20 (SEQ ID NO: 43), G30 (SEQ ID NO: 65) and G40 (SEQ ID NO: 66), was observed 6 months post immunosuppression administration in patients treated with belatacept or CsA.

The present invention is also based, in part, on the surprising finding that while reactivities to poly-T oligonucleotides were found to be very low or undetectable, reactivities to poly-T oligonucleotides can be increased significantly by the addition of at least one G to either the 5′ or the 3′ end of the poly-T oligonucleotides. This was in marked contrast to the reduction of antibody binding to poly-G by the addition of a single T to the 3′ end of the chain.

Thus, quantification of antibodies to particular oligonucleotides (e.g., poly-guanine oligonucleotides or poly-thymine oligonucleotide with at-least one G contiguous to either the 5′ or the 3′), may be used in various embodiments to determine the immunological competence or state of immunosuppression in the individual.

According to a first aspect, the present invention provides a method of determining a level of immunological competence of a subject, the method comprising: obtaining a sample from the subject; assaying the sample for the presence of antibodies to at least one oligonucleotide antigen comprising at least 10 contiguous guanine nucleotides, thereby determining the reactivity of the antibodies in the sample to the at least one oligonucleotide antigen; and comparing the antibody reactivity to the reactivity of a control subject or a reference control value; wherein an equal or higher reactivity of the antibodies in the sample obtained from the subject compared to the reactivity of the control subject is an indication that the subject has a competent immunological system, and wherein a significantly lower reactivity of the antibodies in the sample obtained from the subject compared to control is an indication that the subject has an incompetent immunological system.

In certain embodiments, the method of determining a level of immunological competence of a subject or lack thereof comprises assaying for the presence of antibodies in a sample obtained from the subject to at least one antigen comprising an oligonucleotide sequence comprising at least 10 contiguous guanine nucleotides, thereby determining the reactivity of the antibodies in the sample to the at least one antigen and comparing the antibody reactivity to the reactivity of a control subject or a reference control value, wherein an equal or higher reactivity of the antibodies in the sample obtained from the subject compared to the reactivity of the control subjects is an indication that the subject has a competent immunological system, and wherein a significantly lower reactivity of the antibodies in the sample obtained from the subject compared to the reactivity of the control subjects is an indication that the subject has an incompetent immunological system.

According to a related aspect, the present invention provides a method of determining immunological competence or immunological incompetence of a subject, the method comprising assaying for the presence of antibodies in a sample obtained from the subject to at least one oligonucleotide antigen comprising at least 10 contiguous guanine nucleotides, thereby determining the reactivity of the antibodies in the sample to the at least one oligonucleotide antigen, and comparing the antibody reactivity to the reactivity of a control subject or a reference control value, wherein an equal or higher reactivity of the antibodies in the sample obtained from the subject compared to the reactivity of the control subject is an indication that the subject has a competent immunological system, and wherein a significantly lower reactivity of the antibodies in the sample obtained from the subject compared to control is an indication that the subject has an incompetent immunological system.

According to a further related aspect, the present invention provides a method of determining immunological competence or suppression of a subject, the method comprising obtaining a sample from the subject, assaying the sample for the presence of antibodies to at least one oligonucleotide antigen comprising at least 10 contiguous guanine nucleotides, thereby determining the reactivity of the antibodies in the sample to the at least one oligonucleotide antigen, and comparing the antibody reactivity to the reactivity of a control subject or a reference control value, wherein an equal or higher reactivity of the antibodies in the sample obtained from the subject compared to the reactivity of the control subject is an indication that the subject has a competent immunological system, and wherein a significantly lower reactivity of the antibodies in the sample obtained from the subject compared to control is an indication that the subject has an incompetent immunological system.

It should be understood that the terms “comprise”, “comprises” and “comprising” shall be construed broadly, as if followed by “without limitation”. If A comprises B, then A includes B and may include at least one other component. These terms include also embodiments wherein these terms mean “consist of”, “consists of” and “consisting of”, respectively. If A consists of B, then A includes B and may not include any other component.

The terms “immunological competence” and “immunological incompetence” as used herein refer to the normal or subnormal immunological state of a subject, respectively, as compared to a healthy control subject or a group of healthy control subjects. In certain embodiments, the immune system of immunologically-competent subjects has the ability to develop a normal, healthy immune response towards non-self antigens, such as those found on non-HLA-matching cells or organs, or such as those found on parasites and pathogens. In certain embodiments, patients developing an immune response such as transplant rejection are considered immunologically-competent. In certain embodiments, the immune system of immunologically-incompetent subjects does not have the ability to develop a normal, healthy immune response towards non-self antigens, such as those found on non-HLA-matching cells or organs, or such as those found on parasites and pathogens. In certain embodiments, patients unable to develop an immune response such as transplant rejection are considered immunologically-incompetent. It should be understood that a variety of factors, alone or in combination, may cause or lead subjects to a state of immunological-incompetence. For example, drugs, medical treatments, exposure to radiation, genetics, nutritional condition (including vitamin deficiency), psychiatric condition and even poisoning by certain agents are all major factors on immunological competence.

The terms “immunological suppression” and “immunosuppression” as used herein refer to certain conditions of immunological incompetence in which the subnormal immunological state of a subject is elicited by administration of one or more immunosuppressive agents or treatments. In certain embodiments, such subjects have reduced immune responses toward infections and/or diseases. In certain embodiments, the immune system of immunologically-suppressed subjects does not have the ability to develop a normal immune response towards non-self antigens, such as those found on non-HLA-matching cells or organs, or such as those found on parasites and pathogens. In certain embodiments, immunologically-suppressed patients are unable to develop an immune response such as a transplant rejection.

The term “assaying” as used herein refers to qualitative or quantitative analyzing the presence or absence of a particular entity, such as an interaction or a complex between antibodies and antigens, in a particular setting, such as in a test tube or in an antigen probe set.

The term “antigen” is used herein to refer to a molecule or a portion of a molecule capable of being bound by an antibody. The antigen is typically capable of inducing an animal to produce antibody capable of binding to an epitope of that antigen. An antigen may have one or more epitopes. The specific reaction referred to above is meant to indicate that the antigen will react, in a highly selective manner, with its corresponding antibody and not with the multitude of other antibodies which may be evoked by other antigens. An “antigenic oligonucleotide” is an oligonucleotide which is capable of specifically binding an antibody. The term “oligonucleotide antigen” according to the present invention refers to a nucleotide sequence, consisting between 10 to 50 consecutive nucleotides.

The terms “healthy control subjects”, “healthy subjects” and “control subjects” are used herein to refer to one or more healthy immunocompetent subjects.

The term “competent immunological system” as used herein refers to an immunological system having normal, healthy immune responses. In certain embodiments, normal immune responses lead to low prevalence of infections and/or diseases. In certain embodiments, patients having a competent immunological system are capable of developing normal immune responses such as rejection of a transplant or an organ from a non-HLA-matching donor.

The term “incompetent immunological system” as used herein refers to an immunological system having subnormal, aberrant immune responses. In certain embodiments, subnormal immune responses lead to high prevalence of infections and/or diseases. In certain embodiments, patients having an incompetent immunological system are incapable of developing normal immune responses such as rejection of a transplant or an organ from a non-HLA-matching donor. The term “subnormal” as used herein in the context of the immune response refers to a statistically significant impairment or in other embodiments to a significant impairment as recognized by the skilled artisan e.g. the treating physician.

The term “suppressed immunological system” as used herein refers to patients having an incompetent immunological system due to being treated by immunosuppressive drugs or therapies.

The term “sample” as used herein refers to any composition comprising a biological material obtained or derived from a subject. Non-limiting examples of samples according to the present invention are any kind of a biological tissue or a fluid which comprises antibodies.

According to some embodiments, the subject is a transplant patient or an organ recipient or is being evaluated as an organ recipient candidate (i.e., a transplant candidate). The term “transplant patient” as used herein generally refers to any subject considered for, being evaluated as, or have already been transplanted with any non-self, biological or mechanical device, tissue or organ. A non-limiting example of a transplant patient is a subject implanted with a mechanical pacemaker. The term “organ recipient” as used herein generally refers to any subject considered for, being evaluated as, or have already been transplanted with any non-self, biological or mechanical organ, or a portion of an organ. A non-limiting example of an organ recipient is a subject implanted with a liver, a lobe of a liver or a porcine heart valve. According to additional embodiments, the subject is or was receiving at least one immunosuppressive drug or an immunosuppressive treatment. The phrase “was receiving” as used herein refers to a period of time in which immunosuppressive treatment may still have an effect on the immune system of the subject. In certain embodiments, the subject was receiving immunosuppressive treatment in the year before determining his immunological competence. In certain embodiments, the subject was receiving immunosuppressive treatment at least 1, 2, 3, 4, 5 or 6 months before determining his immunological competence. Each possibility is a separate embodiment of the invention.

According to some embodiments, the at least one immunosuppressive treatment is irradiation. In certain embodiments, the irradiation is a form of radiotherapy. For example, total body irradiation (TBI) is a form of radiotherapy used primarily as part of the preparative regimen for haematopoietic stem cell (or bone marrow) transplantation. Total body irradiation in the setting of bone marrow transplantation serves to destroy or suppress the recipient's immune system, preventing immunologic rejection of transplanted donor bone marrow or blood stem cells. Doses of total body irradiation used in bone marrow transplantation typically range from 10 to >12 Gy. For reference, a dose of 4.5 Gy is fatal in 50% of exposed individuals without aggressive medical care. In certain embodiments, the subject is or was receiving an irradiation dosage of at least 2, at least 4, at least 6, at least 8, at least 10 or at least 12 in total, or per irradiation session.

According to some embodiments, the at least one immunosuppressive drug is selected from the group consisting of: corticosteroids, calcineurin inhibitors (CNIs) including but not limited to cyclosporine or tacrolimus, T-cell costimulatory blocker such as belatacept, purine metabolism inhibitors including but not limited to azathioprine or mycophenolate mofetil (MMF), rapamycins including but not limited to sirolimus or everolimus, immunosuppressive Igs including but not limited to antilymphocyte globulin (ALG) and antithymocyte globulin (ATG), monoclonal antibodies (mAbs) including but not limited to OKT3 or anti-IL-2 receptor monoclonal antibodies. In certain embodiments, the at least one immunosuppressive drug is a T-cell costimulatory blocker, such as belatacept. In certain embodiment, the at least one immunosuppressive drug is a calcineurin inhibitor, such as cyclosporine A. In certain embodiments, the at least one immunosuppressive drug is belatacept. In certain embodiment, the at least one immunosuppressive drug is cyclosporine A.

According to another embodiment, the subject receiving at least one immunosuppressive drug has an autoimmune disease. For a disease to be regarded as an autoimmune disease it needs to answer to Witebsky's postulates: direct evidence from transfer of pathogenic antibody or pathogenic T cells, indirect evidence based on reproduction of the autoimmune disease in experimental animals, circumstantial evidence from clinical clues, and/or genetic architecture clustering with other autoimmune diseases. Non-limiting examples of autoimmune diseases are lupus, multiple sclerosis, rheumatoid arthritis or Crohn's disease. According to another embodiment, the subject is an immunocompromised subject, such as a subject undergoing chemotherapy treatment, a subject afflicted with cancer including but not limited to leukemia, lymphoma, multiple myeloma, a subject afflicted by an immune deficiency disorder, or a subject afflicted with a chronic infections such as acquired immunodeficiency syndrome (AIDS). Each possibility represents a separate embodiment of the present invention. The term “immunocompromised subject” as used herein refers to a subject having subnormal, low, defective or no immunological response compared to a healthy control subject. According to certain embodiments, the subject is not afflicted with systemic lupus erythematosus.

In certain embodiments, the subject is or has been in a state of malnutrition. The terms “malnutrition” and “malnourishment” as used herein refer to a condition that results from eating a diet in which nutrients are not enough or are too much such that it causes health problems. The nutrients involved can include: calories, protein, carbohydrates, vitamins or minerals. It is often used specifically to refer to under-nutrition where the patient is not receiving enough calories, proteins and/or micronutrients; however, it also includes over-nutrition. In certain embodiments, the subject is of a certain genetic profile causing a state of immunological-incompetence. In certain embodiments, the subject is of a certain familial history, determined to be in a state of immunological-incompetence. In certain embodiments, the subject is of a certain psychiatric state or condition causing a state of immunological-incompetence. Each possibility represents a separate embodiment of the invention.

According to some embodiments, the reactivity of antibodies is selected from IgG reactivities and IgM reactivities. In one embodiment, the reactivity of antibodies is IgG reactivities. In another embodiment, the reactivity of antibodies is IgG reactivities.

In certain embodiments, the method of the present invention is indicative to a change of at least 19%, at least 28%, at least 30%, at least 33%, at least 37%, at least 41%, at least 42%, at least 47% or at least 55% in the level of the immunological competence of a subject, when comparing the antibody reactivity to the reactivity of a control subject or a reference control value. In certain embodiments, the method of the present invention is indicative to a change of at least 28%, at least 37%, at least 41%, at least 55% in the level of the immunological competence of a subject, when comparing the IgM antibody reactivity to the reactivity of a control subject or a reference control value. In certain embodiments, the method of the present invention is indicative to a change of at least 19%, at least 30%, at least 33%, at least 42 or at least 47% in the level of the immunological competence of a subject, when comparing the IgG antibody reactivity to the reactivity of a control subject or a reference control value. Each possibility represents a separate embodiment of the invention.

The nomenclature used to refer to the oligonucleotide sequence of each oligonucleotide antigen disclosed in the present invention is as follows: an oligonucleotide antigen consisting of the oligonucleotide sequence of X₂Y₃Z₂, i.e. two oligonucleotides of X followed by three oligonucleotides of Y followed by two oligonucleotides of Z is labeled as X2Y3Z2, (X)2(Y)3(Z)2, or XXYYYZZ, or referred to by its corresponding SEQ ID NO. It should be understood that in this example, X, Y and Z may relate to more than one oligonucleotide, e.g. to 2-20 oligonucleotides. Therefore, an oligonucleotide antigen consisting of the oligonucleotide sequence of X₂, wherein X is a stretch of e.g. two oligonucleotides, e.g. YZ, is labeled as X2, (X)2, or YZYZ, or referred to by its corresponding SEQ ID NO.

According to another embodiment, the at least one oligonucleotide antigen is selected from the group consisting of G14 having the nucleotide sequence as set forth in SEQ ID NO: 41, G20 having the nucleotide sequence as set forth in SEQ ID NO: 43, G40 having the nucleotide sequence as set forth in SEQ ID NO: 66, G17 having the nucleotide sequence as set forth in SEQ ID NO: 36, and G30 having the nucleotide sequence as set forth in SEQ ID NO: 65. In certain embodiments, the at least one antigen is G14 having the nucleotide sequence as set forth in SEQ ID NO: 41. In certain embodiments, the at least one antigen is G20 having the nucleotide sequence as set forth in SEQ ID NO: 43.

In one embodiment, the at least one oligonucleotide antigen is G14 having the nucleotide sequence as set forth in SEQ ID NO: 41. In one embodiment, the at least one oligonucleotide antigen is G20 having the nucleotide sequence as set forth in SEQ ID NO: 43. In one embodiment, the at least one oligonucleotide antigen comprises the nucleotide sequence as set forth in SEQ ID NO: 43 (GGGGGGGGGGGGGGGGGGGG). In an exemplary embodiment, the at least one oligonucleotide antigen consists of the nucleotide sequence as set forth in SEQ ID NO: 43. In another embodiment, the oligonucleotide sequence further comprises at least one thymine at the 5′ termini of the oligonucleotide sequence. In one embodiment, the at least one oligonucleotide antigen comprises the nucleotide sequence as set forth in SEQ ID NO: 38 (TGGGGGGGGGGGGGGGG).

According to some embodiments, the method comprises determining the reactivity of antibodies in the sample obtained from the subject to at least two different oligonucleotide antigens. According to some embodiments, the at least two different oligonucleotide antigens are selected from the group consisting of G14 having the nucleotide sequence as set forth in SEQ ID NO: 41, G20 having the nucleotide sequence as set forth in SEQ ID NO: 43, G40 having the nucleotide sequence as set forth in SEQ ID NO: 66, G17 having the nucleotide sequence as set forth in SEQ ID NO: 36, and G30 having the nucleotide sequence as set forth in SEQ ID NO: 65. In some embodiments, the method comprises determining the reactivity of antibodies in the sample obtained from the subject to G20 antigen (SEQ ID NO: 43) and at least one additional antigen selected from SEQ ID NO: 36, 41, 65 and 66. According to additional embodiments, the method comprises determining the reactivity of antibodies in the sample obtained from the subject to at least three, at least four, at least five or more antigens.

According to another embodiment, the method of the invention comprises determining the reactivity of antibodies in a sample obtained from the subject to an oligonucleotide sequence comprising alternating cytosine-guanine di-nucleotides. In one embodiment, the cytosine-guanine di-nucleotide sequence is a (CG)10 antigen. A (CG)10 antigen, as used herein refers to an oligonucleotide sequence comprising 10 alternating cytosine-guanine di-nucleotides (CGCGCGCGCGCGCGCGCGCG; SEQ ID NO: 25). In a particular embodiment, the method comprises determining IgM reactivities to SEQ ID NO:25. In another embodiment, the cytosine-guanine di-nucleotide antigen comprises the nucleotide sequence as set forth in SEQ ID NO:67 (GCGCGCGCGCGCGCGCGCGC).

According to certain embodiments, the methods, antigen probe sets, arrays or kit of the invention, the oligonucleotide antigens comprises a thymine oligonucleotide sequence comprising at-least one guanine nucleotide contiguous to at least one of the oligonucleotide's termini. The methods of the invention, in some embodiments, comprises determining the reactivity of antibodies in a sample obtained from the subject to an oligonucleotide sequence of at least 10, at least 11, at least 12, at least 13, at least 14, at least 15 or at least 16 thymine nucleotides comprising at-least one guanine nucleotide contiguous to at least one of the oligonucleotide's termini.

In one embodiment, the at least one guanine nucleotide is contiguous to the 5′ end of the thymine oligonucleotide sequence. In another embodiment, the at-least one guanine nucleotide is contiguous to the 3′ end of the thymine oligonucleotide sequence. As use herein “at least one guanine nucleotide” includes but is not limited to 1, 2, 3, 4, 5, 6, 7, 8, 9 or 10 guanine nucleotide, wherein each possibility is a separate embodiment of the present invention.

It should be appreciated that the methods, an antigen probe set, array or kit of the invention the oligonucleotide antigens comprises or includes various oligonucleotide lengths, including at least 10, at least 11, at least 12, at least 13, at least 14, at least 15, at least 16, at least 17, at least 18, at least 19 or at least 20 nucleotides.

In additional embodiments of the methods, antigen probe sets, arrays or kits of the invention, the oligonucleotide antigens comprises at most 50, at most 49, at most 48, at most 47, at most 46, at most 45, at most 44, at most 43, at most 42, at most 41, at most 40, at most 39, at most 38, at most 37, at most 36, at most 35, at most 34, at most 33, at most 32, at most 31, at most 30, at most 29, at most 28, at most 27, at most 26, at most 25, at most 24, at most 23, at most 22, at most 21, at most 20 contiguous guanine nucleotides.

In certain embodiment, the guanine oligonucleotide antigen of the invention comprises at least 14, at least 17, at least 20, at least 30 or at least 40 consecutive guanine nucleotides. In another embodiment the guanine oligonucleotide antigen of the invention comprises at least 10, at least 11, at least 12, at least 13, at least 14, at least 15, at least 16, at least 17, at least 18, at least 19 or at least 20 consecutive guanine nucleotides. In another embodiment the guanine oligonucleotide antigens comprise at most 50, at most 49, at most 48, at most 47, at most 46, at most 45, at most 44, at most 43, at most 42, at most 41, at most 40, at most 39, at most 38, at most 37, at most 36, at most 35, at most 34, at most 33, at most 32, at most 31, at most 30, at most 29, at most 28, at most 27, at most 26, at most 25, at most 24, at most 23, at most 22, at most 21, at most 20 consecutive guanine nucleotides.

In certain embodiments, the oligonucleotide antigen comprises or consists 10-50 contiguous guanine nucleotides. In certain embodiments, the oligonucleotide antigen comprises or consists 10-40 contiguous guanine nucleotides. In certain embodiments, the oligonucleotide antigen comprises or consists 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 contiguous guanine nucleotides. Each possibility represents a separate embodiment of the present invention. It should be understood that since each oligonucleotide antigen according to the present invention comprises at least 10 contiguous guanine nucleotides, and is limited in length to a maximum of 50 nucleotides, room is left for additional oligonucleotide sequences, up to 40 nucleotides in length. In certain embodiments, the additional oligonucleotide sequences are selected from the group consisting of nucleotide sequence as set forth in any one of SEQ ID NOs: 1 to 67. In certain embodiments, the oligonucleotide antigen comprises or consists of at least two sequences selected from the group consisting of nucleotide sequence set forth in any one of SEQ ID NOs: 1 to 67.

In another embodiment the thymine oligonucleotide antigen of the invention (comprising at-least 1 and preferably at least 10 guanine nucleotide at the 5′ or ′3) comprises at least 10, at least 11, at least 12, at least 13, at least 14, at least 15, at least 16 or at least 17 thymine nucleotides. In another embodiment the thymine oligonucleotide antigens comprise at most 50, at most 49, at most 48, at most 47, at most 46, at most 45, at most 44, at most 43, at most 42, at most 41, at most 40, at most 39, at most 38, at most 37, at most 36, at most 35, at most 34, at most 33, at most 32, at most 31, at most 30, at most 29, at most 28, at most 27, at most 26, at most 25, at most 24, at most 23, at most 22, at most 21, at most 20, at most 19, at most 18 or at most 17 thymine nucleotides.

Immunosuppression

In some embodiments, the invention provides a method for determining immunosuppression in an individual. The invention can be used to determine immunosuppression in any individual undergoing any type of immunosuppression with any immunosuppressive agent or treatment. In one embodiment, the method further comprises modifying immunosuppression dosing for the individual subsequent to determining insufficient or excessive immunosuppression.

In connection with immunosuppression, a wide variety of immunosuppressive agents is known in the art and is routinely administered to individuals for a variety of purposes. The invention is suitable for determining immunosuppression in any individual undergoing any type of immunosuppression with any immunosuppressive agent, which include but are not limited to calcineurin inhibitors (CNI) (i.e., tacrolimus or cyclosporine), antilymphocyte drugs such as OKT3, Antithymocytegamma globulin (ATGAM), Daclizumab, and Basiliximab (anti IL2R); antimetabolites such as Azathioprine, Cyclophosphamide, and Mycophenolate mofetil; mammalian Target Of Rapamycin (mTOR) inhibitors such as Sirolimus (Rapamune), or corticocorticoids such as Prednisone, or methylprednisolone (Solumedrol). In exemplified embodiments, the at least one immunosuppressive drug is a T-cell costimulatory blocker such as belatacept. In another exemplified embodiment, the at least one immunosuppressive drug is a calcineurin inhibitor such as cyclosporine A. It is considered that the method is suitable for evaluating the immunosuppression status of any mammal, including humans, and ranging in age from infants to the elderly.

Determining an amount of anti-guanine oligonucleotide antibodies in a sample obtained from an individual, wherein the amount is different from a control, is considered to be indicative that the individual is a candidate for an alteration of his or her immunosuppression therapy. For example, an individual for whom performing the method of the invention indicates insufficient immunosuppression could be recommended for an increase in dosing, or for a change to a different immunosuppression agent. Likewise, an individual for whom performing the method of the invention indicates excessive immunosuppression could be recommended for a decrease in dosing, or for a change to a different immunosuppression agent. An individual for whom performing the method of the invention indicates an appropriate amount of immunosuppression could be recommended for no change in immunosuppression regime.

The method of the invention can be repeated to monitor the immunosuppression status of an individual over time. For example, the invention can be used in order to monitor the impact of a course of events on an individual's immune system and/or evaluate whether modifications of the immunosuppression therapy of an individual should be considered and/or implemented. The method of the invention can also be performed prior to initiation of immunosuppression therapy and compared to a sample(s) of blood obtained from the individual after initiation of immunosuppression therapy to evaluate the efficacy of the therapy. In some embodiments, the method of the invention can be performed before and after transplant surgery is performed, in order to monitor changes over time in the immune response of the patient in response to these medical procedures. This information regarding the patient's immune status may be useful as an adjunct to therapeutic drug monitoring at any point in the course of therapy in order to assess the progress of a patient, the suitability of a drug regimen, and to predict clinical outcomes for a patient.

In additional embodiments, the present invention provides methods of determining and monitoring the state of a patient's immune system without having to stimulate the system; both IgG and IgM antibodies to guanine oligonucleotides are produced spontaneously.

In some embodiments, the patient is one who is receiving or will be receiving an immuno-modulating drug or treatment. For example, the patient may be the recipient of an organ such as heart, lungs, kidney, pancreas, liver, bowel, skin, bone marrow cartilage, ligament, muscle or other organs. In an embodiment, the patient is a kidney recipient. In another embodiment, the patient is a kidney recipient receiving an immunosuppressive drug selected from a T-cell co-stimulatory blocker such as belatacept or a calcineurin inhibitor such as cyclosporine A. Further, a transplant patient may be the recipient of more than one organ, e.g. a “heart-lung” transplant recipient. Alternatively, the transplant may be transplanted tissue. The transplanted tissue or organ(s) may be from any source known to those of skill in the art, for example, from a live organ donor such as a relative (e.g. a sibling) or a matched non-related donor; from a cadaver; or from a tissue or artificial “organ” that has been developed and/or maintained in a laboratory setting, e.g. tissue or “organs” grown from stem cells, or cultured in a laboratory setting from tissue or cell samples. The transplant may also be a xenographic transplant, or a transplantation of a synthetic substance.

Alternatively, the patient may be under treatment for an autoimmune disease. Examples of autoimmune diseases are abundant, such as rheumatoid arthritis, lupus, Crohn's disease, psoriasis, etc. In other embodiments, the patient may be afflicted with an infectious disease, such as Human Immune Deficiency Syndrome related viruses (HIV-1), or Hepatitis associated viruses (HCV). Further, the patient may be a cancer patient. Those of skill in the art will recognize that the methods of the present invention may be used to monitor and/or assess the immune system of any individual for any reason.

In yet another embodiment, the invention provides a method for assessing the pharmacodynamic impact of an immunosuppressant drug in a non-transplant patient. The non-transplant patient may be receiving the immunosuppressant drug for a disease condition including but not limited to autoimmunity, inflammation, Crohn's Disease, lupus erythematosus, or rheumatoid arthritis. The method will typically be carried out in order to reduce complications from infections or cancer in the non-transplant patient

The present invention provides a method of guiding decisions regarding therapies and of predicting a clinical outcome of a patient receiving one or more immunosuppressive drugs or treatments. Possible clinical outcomes include, for example, rejection of the transplanted organ, infection, or organ toxicity. In order to predict clinical outcomes such as these, it is advantageous to determine an initial level of the subject's antibodies to guanine oligonucleotides as early in the immunosuppressive drug course as possible in order to start surveillance of the patient's immune status coincident with or soon after transplant surgery, but monitoring may begin at any point after the administration of the immuno-modulating drugs. Subsequent immune responses are ascertained and compared to the earlier response, and to each other. Any given immune response value (e.g., IgG of IgM reactivity levels to guanine oligonucleotides) can be assigned to a category of a known range of values, and a comparison of changes in measured values over time allows the observation of trends in the immune response of the patient.

In another embodiment, an initial sample (e.g., blood sample) is obtained and tested prior to organ transplant surgery and before any immunosuppressant drug is administered. The anti-oligonucleotide antibody value is ascertained and compared to the categories of known value ranges (e.g., low, moderate or strong). Based on these values the initial drug dose may be maintained within or modified from the usual practice of dose assignment on the basis of patient body weight. For example, a transplant candidate who is determined to be immunosuppressed or immuno-incompetent due to an immuno-suppressing disease or infectious disease (e.g. AIDS) may be given a lower or no drug dose, compared to another individual of the same body weight.

In another preferred embodiment, an initial blood sample is obtained and tested prior to organ transplant surgery and before any immunosuppressant drug is administered, and another blood sample is tested after surgery and after the administration of drugs. By comparing the values obtained from these samples, medical judgments can be made relative to the effect of the surgery and drugs on the patient specifically regarding the immune status. For example, if the value obtained from the sample obtained subsequent to the first was in a lower range than the first, additional testing may be indicated and or medication doses reduced to avoid the possibility of over-medication. If the value obtained from the sample obtained subsequent to the first one was in a higher range than the first, additional testing may be indicated and or medication doses increased to avoid the possibility of organ rejection.

In another preferred embodiment, a blood sample obtained and tested at any point after surgery can provide immune status information regarding the level of immune suppression when the values are compared to categories of known value ranges. For example, if the value obtained is in the weak range, additional testing maybe indicated and or medication doses reduced to avoid the possibility of over medication. If the value obtained is in the strong range, additional testing may be indicated and/or medication doses increased, or rescue therapy initiated to avoid the possibility of organ rejection. Further, if the value is in the moderate range, and particularly if the value does not fluctuate significantly (e.g. stays within the same range) for at least two consecutive monthly measurements, this may indicate that stability of the immune response has been achieved, and that adjustments to the treatment regimen are not necessary at that time.

Regarding the frequency at which samples (e.g., blood samples) are analyzed, those of skill in the art will recognize that sampling may be done at any point at which a skilled practitioner (e.g. a physician) deems it to be advisable. In general, such testing would be carried out at most daily (e.g. during a time when a patient is most at risk) and at least monthly (e.g. during a time when a patient appears to be relatively stable); it should be noted that the half-life in humans of IgM is approximately 5 days and the half-life of IgG is about 7-21 days depending on the isotype.

In yet another preferred embodiment, multiple samples are obtained and tested at multiple points after the organ transplant surgery and during the period when immuno-modulating drugs are being administered. An example of the predictive value of the methods would be the detection, by utilizing the methods of the present invention, of an increase in the immune response of the patient from the low to moderate to the strong range over a period of time. The results may be predictive of potential acute rejection of the transplanted organ, and may warrant, for example: initiation of other confirmatory tests (e.g. organ biopsy or organ specific blood chemistry analyses); an increase in the dose of the drug being administered; a rescue therapy with an alternate drug; or a new combination of drugs. In general, in order to predict potential organ rejection, the state of the immune response must be monitored for several days, and preferably for about 3-10 days.

On the other hand, an unexpected decrease in the immune response over a period of time may be predictive of the risk of developing an opportunistic infection due to over medication. For example, if a patient's immune response declines from the moderate range to the low range, this may be indicative of over-medication and warrant the initiation of further confirmatory tests (e.g. organ biopsy or organ function analysis, or assays for infectious organisms by PCR), or a reduction or change in medication. In general, in order to detect possible over medication, the state of the immune response must be monitored for several days, and preferably for about 3-10 days.

The method may further be useful for monitoring a patient's immune response during the standard immuno-suppressive-therapy phase of “weaning” the patient from the drugs, i.e. the phase during which a patient's drug dosage is lowered as much as possible to reduce the risk of toxicity, while maintaining a low chance of transplant rejection. In particular this assay is especially valuable for monitoring tolerance protocols where the objective is the eventual removal of all immunosuppressive drugs. Similarly, the method may also be used to assess patient compliance with prescribed medication regimens.

The method is also of value in monitoring the functional status of the immune responses of long-term organ recipients, who have been on the same medication dosages for extended time periods (e.g., years). Patients who have taken immunosuppressive drugs over a long period have been shown to suffer from over suppression concurrent with extended drug courses.

As used herein, the “reactivity of antibodies in a sample” or the “reactivity of an antibody in a sample” to “an antigen” or “a plurality of antigens” refers to the immune reactivity of each antibody in the sample to a specific antigen, potentially selected from the plurality of antigens. The immune reactivity of the antibody to the antigen, i.e. its ability to specifically bind the antigen, may be used to determine the amount of the antibody in the sample. The calculated levels of each one of the tested antibodies in the sample are collectively referred to as the reactivity pattern of the sample to these antigens. The reactivity pattern of the sample reflects the levels of each one of the tested antibodies in the sample, thereby providing a quantitative assay. In a preferred embodiment, the antibodies are quantitatively determined.

Unless otherwise indicated, the term “plurality” as used herein refers to a group of three or more members. For example, a plurality of antigens means at least three antigens. A plurality, according to the present inventions, further means at least 2, at least 4, at least 10, at least 50, at least 100, at least 150, at least 200, or more.

A “significant difference” between reactivity patterns refers, in different embodiments, to a statistically significant difference, or in other embodiments to a significant difference as recognized by a skilled artisan. In yet another preferred embodiment, a significant (quantitative) difference between the reactivity pattern of the sample obtained from the subject compared to the control reactivity pattern is an indication that the subject has a suppressed immunological system. In specific embodiments, up-regulation or enhanced reactivity of the reactivity of an antibody in a sample to an antigen refers to an increase (i.e., elevation) of about at least two, about at least three, about at least four, or about at least five times higher (i.e., greater) than the reactivity levels of the antibody to the antigen in the control. In another embodiment, down-regulation or decreased reactivity of the reactivity of an antibody in a sample to an antigen refers to a decrease (i.e., reduction) of about at least two, about at least three, about at least four, or about at least five times lower than the reactivity levels of the antibody to the antigen in the control.

In particular embodiments, the significant difference is determined using a cutoff of a positive predictive value (PPV) of at least 85%, preferably at least 90%. Determining a PPV for a selected marker (e.g., an antigen) is well known to the ordinarily skilled artisan and is exemplified in the methods described below. Typically, positivity for an antigen is determined if it detected above 10% of the subjects in a specific study subgroup using a selected cutoff value, such as PPV≧90%. For example, antigen i is determined to specifically characterize group A if it detected at least 10% of the subjects in group A with a PPV≧90% when compared to a different test group B. Subjects in group A that are above the cutoff of PPV≧90% for antigen i are considered to be positive for antigen i.

An antibody “directed to” an antigen, as used herein is an antibody which is capable of specifically binding the antigen. Determining the levels of antibodies directed to a plurality of antigens includes measuring the level of each antibody in the sample, wherein each antibody is directed to a specific antigen of the invention. This step is typically performed using an immunoassay, as detailed herein.

In other embodiments, determining the reactivity of antibodies in the sample (obtained from the subject) to the plurality of antigens, (and the levels of each one of the tested antibodies in the sample) is performed by a process comprising (i) contacting the sample, under conditions such that a specific antigen-antibody complex may be formed, with an antigen probe set comprising the plurality of antigens, and (ii) quantifying the amount of antigen-antibody complex formed for each antigen probe. The amount of antigen-antibody complex is indicative of the level of the tested antibody in the sample (or the reactivity of the sample with the antigen).

In another embodiment the method comprises determining the reactivity of at least one IgG antibody and at least one IgM antibody in the sample to the plurality of antigens. In another embodiment, the method comprises determining the reactivity of a plurality of IgG antibodies and at least one IgM antibodies in the sample to the plurality of antigens.

One of the major advantages provided by the present invention is the provision of novel indicative oligonucleotide antigens, and novel subsets of indicative oligonucleotide antigens. It is the binding of a subject's antibodies to these indicative oligonucleotide antigens which is indicative to the subject's immunological competence, without any dependency of the subject's identity or medical history Importantly, these novel indicative oligonucleotide antigens cancel the historic need to identify specific markers in each subject individually to follow his immunological competence. Advantageously, the method provided by the present invention may be performed in very small scale, using only one oligonucleotide antigen, in e.g. a single receptacle. For example, in some embodiments, the method may be performed in a single receptacle, such as a test tube or a plate. In certain embodiments, the method may be performed by a dipstick, to which the oligonucleotide antigen is attached. Typically, determining the reactivity of antibodies in the sample to the plurality of antigens is performed using an immunoassay. Advantageously, in other certain embodiments, a plurality of antigens may be used in the form of a probe set, an antigen array or an antigen chip.

Antigen Probes and Antigen Probe Sets

According to further embodiments, the invention provides antigen probes and antigen probe sets useful for determining immunological competence or lack thereof, as detailed herein.

According to the principles of the invention, the invention further provides a plurality of antigens also referred to herein as antigen probe sets or sets of antigen probes. According to the principles of the invention, the plurality of antigens may advantageously be used in the form of an antigen array. According to some embodiments the antigen array is conveniently arranged in the form of an antigen chip.

A “probe” as used herein means any compound capable of specific binding to a component. According to one aspect, the present invention provides an antigen probe set comprising a plurality of antigens of the antigens of the invention or any combinations thereof. According to certain embodiments, the antigen probe set comprises a subset of the antigens of the present invention. In another particular embodiment, the subset of antigen is selected from G14 having the nucleotide sequence as set forth in SEQ ID NO: 41, G20 having the nucleotide sequence as set forth in SEQ ID NO: 43, G40 having the nucleotide sequence as set forth in SEQ ID NO: 66, G17 having the nucleotide sequence as set forth in SEQ ID NO: 36, and G30 having the nucleotide sequence as set forth in SEQ ID NO: 65.

The reactivity of antibodies to the plurality of antigens of the invention may be determined according to techniques known in the art.

Antigen probes to be used in the assays of the invention may be synthesized using methods well known in the art. It should be noted, that the invention utilizes antigen probes as well as homologs, fragments and derivatives thereof, as long as these homologs, fragments and derivatives are immunologically cross-reactive with these antigen probes. The term “immunologically cross-reactive” as used herein refers to two or more antigens that are specifically bound by the same antibody. The term “homolog” as used herein refers to an oligonucleotide having at least 80%, at least 85%, at least 90% or at least 98% identity to the antigen's nucleotide sequence. Cross-reactivity can be determined by any of a number of immunoassay techniques, such as a competition assay (measuring the ability of a test antigen to competitively inhibit the binding of an antibody to its known antigen).

The term “fragment” as used herein refers to a portion of a oligonucleotide, or oligonucleotide analog which remains immunologically cross-reactive with the antigen probes, e.g., to immunospecifically recognize the target antigen. The fragment may have the length of about 80%, about 85%, about 90%, at least 95% or about 98% of the respective antigen.

The term “oligonucleotide” as used herein relates to a nucleotide sequence of between 10 and 50 nucleotides in length, alternatively between 14 and 40 nucleotides in length.

According to additional embodiments, the antigen probe set comprises at least 100, at least 150, at least 200 or more antigens, including one or a plurality of the antigens provided by the present invention. According to additional embodiments, the antigen probe set comprises at least 100, at least 150, at least 200 or more oligonucleotide antigens, including one or a plurality of the oligonucleotide antigens provided by the present invention.

In other aspects, there are provided nucleic vectors comprising the oligonucleotides of the invention and host cells containing them. These nucleic acids, vectors and host cells are readily produced by recombinant methods known in the art (see, e.g., Sambrook et al., 2001). A nucleic acid molecule can also be produced using recombinant DNA technology (e.g., polymerase chain reaction (PCR) amplification, cloning) or chemical synthesis. Nucleic acid sequences include natural nucleic acid sequences and homologs thereof, including, but not limited to, natural allelic variants and modified nucleic acid sequences in which nucleotides have been inserted, deleted, substituted, and/or inverted in such a manner that such modifications do not substantially interfere with the nucleic acid molecule's ability to perform the methods of the present invention.

According to the principles of the invention the kits comprise a plurality of antigens also referred to herein as antigen probe sets. These antigen probe sets comprising a plurality of antigens are useful for determining immunological competence or lack thereof. According to the principles of the invention, the plurality of antigens may advantageously be used in the form of an antigen array. According to some embodiments the antigen array is conveniently arranged in the form of an antigen chip.

In other embodiments, the kit may further comprise means for determining the reactivity of antibodies in a sample to the plurality of antigens. For example, the kit may contain reagents, detectable labels and/or containers which may be used for measuring specific binding of antibodies to the antigen probes of the invention. In a particular embodiment, the kit is in the form of an antigen array. In some embodiments, the kit comprises means for comparing reactivity patterns of antibodies in different samples to the plurality of antigens. In other embodiments, the kit may further comprise negative and/or positive control samples.

The phrase “means for determining the reactivity of an antibody to an antigen” as used herein refers to devices, reagents and chemicals, such as vials, buffers and written protocols or instructions, used to perform biological or chemical assays.

For example, a control sample may contain a sample from at least one healthy individual (as a reference for immunological competence). Alternatively, a control may contain a sample from at least one immunological competent individual. Other non-limiting examples are a panel of control samples from a set of healthy individuals or diseased individuals, or a stored set of data from control individuals.

Antibodies, Samples and Immunoassays

Antibodies, or immunoglobulins, comprise two heavy chains linked together by disulfide bonds and two light chains, each light chain being linked to a respective heavy chain by disulfide bonds in a “Y” shaped configuration. Each heavy chain has at one end a variable domain (VH) followed by a number of constant domains (CH). Each light chain has a variable domain (VL) at one end and a constant domain (CL) at its other end, the light chain variable domain being aligned with the variable domain of the heavy chain and the light chain constant domain being aligned with the first constant domain of the heavy chain (CH1). The variable domains of each pair of light and heavy chains form the antigen binding site.

The isotype of the heavy chain (gamma, alpha, delta, epsilon or mu) determines immunoglobulin class (IgG, IgA, IgD, IgE or IgM, respectively). The light chain is either of two isotypes (kappa, κ or lambda, λ) found in all antibody classes.

It should be understood that when the terms “antibody” or “antibodies” are used, this is intended to include intact antibodies, such as polyclonal antibodies or monoclonal antibodies (mAbs), as well as proteolytic fragments thereof such as the Fab or F(ab′)2 fragments. Further included within the scope of the invention (for example as immunoassay reagents, as detailed herein) are chimeric antibodies; recombinant and engineered antibodies, and fragments thereof.

Exemplary functional antibody fragments comprising whole or essentially whole variable regions of both light and heavy chains are defined as follows (i) Fv, defined as a genetically engineered fragment consisting of the variable region of the light chain and the variable region of the heavy chain expressed as two chains; (ii) single-chain Fv (“scFv”), a genetically engineered single-chain molecule including the variable region of the light chain and the variable region of the heavy chain, linked by a suitable polypeptide linker; (iii) Fab, a fragment of an antibody molecule containing a monovalent antigen-binding portion of an antibody molecule, obtained by treating whole antibody with the enzyme papain to yield the intact light chain and the Fd fragment of the heavy chain, which consists of the variable and CH1 domains thereof; (iv) Fab′, a fragment of an antibody molecule containing a monovalent antigen-binding portion of an antibody molecule, obtained by treating whole antibody with the enzyme pepsin, followed by reduction (two Fab′ fragments are obtained per antibody molecule); and (v) F(ab′)2, a fragment of an antibody molecule containing a monovalent antigen-binding portion of an antibody molecule, obtained by treating whole antibody with the enzyme pepsin (i.e., a dimer of Fab′ fragments held together by two disulfide bonds).

In another embodiment, detection of the capacity of an antibody to specifically bind an antigen probe may be performed by quantifying specific antigen-antibody complex formation. The term “specifically bind” as used herein means that the binding of an antibody to an antigen probe is not competitively inhibited by the presence of non-related molecules.

In certain embodiments, the method of the present invention is performed by determining the capacity of an antigen of the invention to specifically bind antibodies of the IgG isotype, or, in other embodiments, antibodies of the IgM, isolated from a subject. Methods for obtaining suitable antibody-containing biological samples from a subject are well within the ability of those of skill in the art. Typically, suitable samples comprise whole blood and products derived therefrom, such as plasma and serum. In other embodiments, other antibody-containing samples may be used, e.g. CSF, urine and saliva samples. Numerous well known fluid collection methods can be utilized to collect the biological sample from the subject in order to perform the methods of the invention. According to certain embodiments, the sample obtained from the subject is selected from the group consisting of serum, plasma and blood. According to one embodiment, the sample is a serum sample.

In certain embodiments, the oligonucleotide antigen consists of an oligonucleotide sequence set forth in SEQ ID NO: 10, 11, 12, 18, 19, 20, 24, 27, 31, 32, 36, 38, 41, 43, 47, 60, 62, 65 or 66. Each possibility represents a separate embodiment of the present invention. In certain embodiments, the oligonucleotide antigen consists of an oligonucleotide sequence selected from the group consisting of the oligonucleotide sequence set forth in SEQ ID NOs: 19, 24, 27, 31, 32, 36, 38, 41, 43, 47, 60, 62, 65 and 66. In certain embodiments, the at least 10 contiguous guanine nucleotides are not attached to a thymine at the 3′ terminus of the contiguous guanine nucleotides. Each possibility represents a separate embodiment of the present invention.

According to some embodiments, the at least one oligonucleotide antigen is used in the form of an antigen probe set. According to some embodiments, the reactivity of antibodies to at least one oligonucleotide antigen is determined using an antigen chip or antigen array.

In certain embodiments, the method comprises determining the reactivity of antibodies in the sample obtained from the subject to at least one antigen selected from the group consisting of G14 having the nucleotide sequence as set forth in SEQ ID NO: 41, G20 having the nucleotide sequence as set forth in SEQ ID NO: 43, G40 having the nucleotide sequence as set forth in SEQ ID NO: 66, G17 having the nucleotide sequence as set forth in SEQ ID NO: 36, and G30 having the nucleotide sequence as set forth in SEQ ID NO: 65; and to at least one antigen selected from the group consisting of antigens having the nucleotide sequence as set forth in any one of SEQ ID NOs: 1 to 67.

According to certain embodiments, the method described above further comprises the step of determining that a subject who has a competent immunological system is not amenable for organ transplantation. According to certain embodiments, the method described above further comprises the step of determining that a subject who has a suppressed or an incompetent immunological system is amenable for organ transplantation. According to certain embodiments, the method described above further comprises the step of determining that a subject who has a competent immunological system and receives immunosuppressive treatment or an immunosuppressive drug is amenable for higher dosages of the immunosuppressive treatment or the immunosuppressive drug than previously administered. According to certain embodiments, the method described above further comprises the step of determining that a subject who has a suppressed or an incompetent immunological system and receiving immunosuppressive treatment or an immunosuppressive drug is amenable for lower dosages of the immunosuppressive treatment or the immunosuppressive drug than previously administered.

According to certain embodiments, the antigen probe described above comprises a plurality of different oligonucleotide antigens selected from the group consisting of G14 having the nucleotide sequence as set forth in SEQ ID NO: 41, G20 having the nucleotide sequence as set forth in SEQ ID NO: 43, G40 having the nucleotide sequence as set forth in SEQ ID NO: 66, G17 having the nucleotide sequence as set forth in SEQ ID NO: 36, and G30 having the nucleotide sequence as set forth in SEQ ID NO: 65.

According to another aspect, the present invention provides an article of manufacture comprising the antigen probe set described above. In some embodiments, the article of manufacture is useful for determining immunological competence of a subject or lack thereof.

In certain embodiments, the article of manufacture is in the form of an antigen probe array or in the form of an antigen chip or in the form of a dipstick or in the form of a lateral flow test or in the form of an ELISA plate or in the form of a Quanterix system or in the form of a dipsticks or any other platform known to those skilled in the art. An “antigen probe array” generally refers to a plurality of antigen probes, either mixed in a single container or arranged in two or more containers. An “antigen chip” generally refers to a substantially two dimensional surface, onto which a plurality of antigens are attached or adhered. A “dipstick” generally refers to an object, onto which a plurality of antigens are attached or adhered, which is dipped into a liquid to perform a chemical test or to provide a measure of quantity found in the liquid. A “lateral flow test” generally refers to devices intended to detect the presence (or absence) of a target analyte in sample (matrix) without the need for specialized and costly equipment.

In certain embodiments, the article of manufacture is in the form of a kit. In certain embodiments, the kit further comprises means for determining the reactivity of antibodies in a sample to at least one oligonucleotide antigen of the antigen probe chip or array. In certain embodiments, the article of manufacture further comprises means for performing the method described above, or instructions for use.

According to another aspect, there is provided the use of the at least one oligonucleotide antigen of the invention for the preparation of an antigen probe set, an antigen probe array, an antigen chip or a kit, optionally for determining immunological competence or immunological incompetence of a subject.

In accordance with the present invention, any suitable immunoassay can be used when performing the methods presented herein. Such techniques are well known to the ordinarily skilled artisan and have been described in many standard immunology manuals and texts. In certain preferable embodiments, determining the capacity of the antibodies to specifically bind the antigen probes is performed using an antigen probe array-based method. Preferably, the array is incubated with suitably diluted serum of the subject so as to allow specific binding between antibodies contained in the serum and the immobilized antigen probes, washing out unbound serum from the array, incubating the washed array with a detectable label-conjugated ligand of antibodies of the desired isotype, washing out unbound label from the array, and measuring levels of the label bound to each antigen probe.

The Antigen Chip

Antigen microarrays are recently developed tools for the high-throughput characterization of the immune response (Robinson et al., 2002, Nat Med 8, 295-301), and have been used to analyze immune responses in vaccination and in autoimmune disorders (Robinson et al., 2002; Robinson et al., 2003, Nat Biotechnol. 21, 1033-9; Quintana et al., 2004; Kanter et al., 2006, Nat Med 12, 138-43). It has been hypothesized, that patterns of multiple reactivities may be more revealing than single antigen-antibody relationships (Quintana et al., 2006, Lupus 15, 428-30) as shown in previous analyses of autoimmune repertoires of mice (Quintana et al., 2004; Quintana et al., 2001, J Autoimmun 17, 191-7) and humans (Merbl et al., 2007, J Clin Invest 117, 712-8; Quintana et at, 2003, J Autoimmun 21, 65-75) in health and disease. Thus, autoantibody repertoires have the potential to provide both new insights into the pathogenesis of the disease and to serve as immune biomarkers (Cohen, 2007, Nat Rev Immunol. 7, 569-74) of the disease process.

According to some aspects the methods of the present invention may be practiced using antigen arrays as disclosed in WO 02/08755 and U.S. 2005/0260770 to some of the inventors of the present invention, the contents of which are incorporated herein by reference. WO 02/08755 is directed to a system and an article of manufacture for clustering and thereby identifying predefined antigens reactive with undetermined immunoglobulins of sera derived from patient subjects in need of diagnosis of disease or monitoring of treatment. Further disclosed are diagnostic methods, and systems useful in these methods, employing the step of clustering a subset of antigens of a plurality of antigens, the subset of antigens being reactive with a plurality of antibodies being derived from a plurality of patients, and associating or disassociating the antibodies of a subject with the resulting cluster.

U.S. Pat. App. Pub. No. 2005/0260770 to some of the inventors of the present invention discloses an antigen array system and diagnostic uses thereof. The application provides a method of diagnosing an immune disease, particularly diabetes type 1, or a predisposition thereto in a subject, comprising determining a capacity of immunoglobulins of the subject to specifically bind each antigen probe of an antigen probe set. The teachings of the disclosures are incorporated in their entirety as if fully set forth herein.

In other embodiments, various other immunoassays may be used, including, without limitation, enzyme-linked immunosorbent assay (ELISA), flow cytometry with multiplex beads (such as the system made by Luminex), surface plasmon resonance (SPR), elipsometry, and various other immunoassays which employ, for example, laser scanning, light detecting, photon detecting via a photo-multiplier, photographing with a digital camera based system or video system, radiation counting, fluorescence detecting, electronic, magnetic detecting and any other system that allows quantitative measurement of antigen-antibody binding.

Various methods have been developed for preparing arrays suitable for the methods of the present invention. State-of-the-art methods involves using a robotic apparatus to apply, print or “spot” distinct solutions containing antigen probes to closely spaced specific addressable locations on the surface of a planar support, typically a glass support, such as a microscope slide, which is subsequently processed by suitable thermal and/or chemical treatment to attach antigen probes to the surface of the support. Conveniently, the glass surface is first activated by a chemical treatment that leaves a layer of reactive groups such as epoxy groups on the surface, which bind covalently any molecule containing free amine or thiol groups. Suitable supports may also include silicon, nitrocellulose, paper, cellulosic supports and the like.

In certain embodiments, the antigen probe set further comprises at least one antigen selected from the group consisting of antigens having the nucleotide sequence as set forth in any one of SEQ ID NOs: 1 to 67. In certain embodiments, the antigen probe set further comprises all of the antigens having the nucleotide sequence as set forth in any one of SEQ ID NOs: 1 to 67.

Preferably, each antigen probe, or distinct subset of antigen probes of the present invention, which is attached to a specific addressable location of the array is attached independently to at least one, at least two, more preferably to at least three separate specific addressable locations of the array in order to enable generation of statistically robust data. In certain embodiments, at least one antigen is attached to at least one specific addressable location of the array. In certain embodiments, one antigen is attached to at least one specific addressable location of the array. In certain embodiments, at least one antigen is attached to one specific addressable location of the array. In an embodiment, one antigen is attached to one specific addressable location of the array.

In addition to antigen probes of the invention, the array may advantageously include control antigen probes or other standard chemicals. Such control antigen probes may include normalization control probes. The signals obtained from the normalization control probes provide a control for variations in binding conditions, label intensity, “reading” efficiency and other factors that may cause the signal of a given binding antibody-probe ligand interaction to vary. For example, signals, such as fluorescence intensity, read from all other antigen probes of the antigen probe array are divided by the signal (e.g., fluorescence intensity) from the normalization control probes thereby normalizing the measurements. Normalization control probes can be bound to various addressable locations on the antigen probe array to control for spatial variation in antibody-ligand probe efficiency. Preferably, normalization control probes are located at the corners or edges of the array to control for edge effects, as well as in the middle of the array.

The labeled antibody ligands may be of any of various suitable types of antibody ligand. Preferably, the antibody ligand is an antibody which is capable of specifically binding the Fc portion of the antibodies of the subject used. For example, where the antibodies of the subject are of the IgM isotype, the antibody ligand is preferably an antibody capable of specifically binding to the Fc region of IgM antibodies of the subject.

The ligand of the antibodies of the subject may be conjugated to any of various types of detectable labels. Preferably the label is a fluorophore, most preferably Cy3. Alternately, the fluorophore may be any of various fluorophores, including Cy5, fluorescein isothiocyanate (FITC), phycoerythrin (PE), rhodamine, Texas red, and the like. Suitable fluorophore-conjugated antibodies specific for antibodies of a specific isotype are widely available from commercial suppliers and methods of their production are well established.

Antibodies of the subject may be isolated for analysis of their antigen probe binding capacity in any of various ways, depending on the application and purpose. While the subject's antibodies may be suitably and conveniently in the form of blood serum or plasma or a dilution thereof (e.g. 1:10 dilution), the antibodies may be subjected to any desired degree of purification prior to being tested for their capacity to specifically bind antigen probes. The method of the present invention may be practiced using whole antibodies of the subject, or antibody fragments of the subject which comprises an antibody variable region.

Data Analysis

The methods of the invention may employ the use of learning and pattern recognition analyzers, clustering algorithms and the like, in order to discriminate between reactivity patterns of healthy control subjects to those of immunosuppressed patients. As such, this term specifically includes a difference measured by, for example, determining the reactivity of antibodies in a test sample to a plurality of antigens, and comparing the resulting reactivity pattern to the reactivity patterns of negative and positive control samples using such algorithms and/or analyzers. The difference may also be measured by comparing the reactivity pattern of the test sample to a predetermined classification rule obtained in such manner.

In some embodiments, the methods of the invention may employ the use of learning and pattern recognition analyzers, clustering algorithms and the like, in order to discriminate between reactivity patterns of immunosuppressed subjects to control subjects. For example, the methods may include determining the reactivity of antibodies in a test sample to a plurality of antigens, and comparing the resulting pattern to the reactivity patterns of negative and positive control samples using such algorithms and/or analyzers.

For example, the algorithm may include, without limitation, supervised or non-supervised classifiers including statistical algorithms including, but not limited to, principal component analysis (PCA), partial least squares (PLS), multiple linear regression (MLR), principal component regression (PCR), discriminant function analysis (DFA) including linear discriminant analysis (LDA), and cluster analysis including nearest neighbor, artificial neural networks, coupled two-way clustering algorithms, multi-layer perceptrons (MLP), generalized regression neural network (GRNN), fuzzy inference systems (FIS), self-organizing map (SOM), genetic algorithms (GAS), neuro-fuzzy systems (NFS), adaptive resonance theory (ART).

In certain embodiments, one or more algorithms or computer programs may be used for comparing the amount of each antibody quantified in the test sample against a predetermined cutoff (or against a number of predetermined cutoffs). Alternatively, one or more instructions for manually performing the necessary steps by a human can be provided.

Algorithms for determining and comparing pattern analysis include, but are not limited to, principal component analysis, Fisher linear analysis, neural network algorithms, genetic algorithms, fuzzy logic pattern recognition, and the like. After analysis is completed, the resulting information can, for example, be displayed on display, transmitted to a host computer, or stored on a storage device for subsequent retrieval.

Many of the algorithms are neural network based algorithms. A neural network has an input layer, processing layers and an output layer. The information in a neural network is distributed throughout the processing layers. The processing layers are made up of nodes that simulate the neurons by the interconnection to their nodes Similar to statistical analysis revealing underlying patterns in a collection of data, neural networks locate consistent patterns in a collection of data, based on predetermined criteria.

Suitable pattern recognition algorithms include, but are not limited to, principal component analysis (PCA), Fisher linear discriminant analysis (FLDA), soft independent modeling of class analogy (SIMCA), K-nearest neighbors (KNN), neural networks, genetic algorithms, fuzzy logic, and other pattern recognition algorithms. In some embodiments, the Fisher linear discriminant analysis (FLDA) and canonical discriminant analysis (CDA) as well as combinations thereof are used to compare the output signature and the available data from the database.

In other embodiments, principal component analysis is used. Principal component analysis (PCA) involves a mathematical technique that transforms a number of correlated variables into a smaller number of uncorrelated variables. The smaller number of uncorrelated variables is known as principal components. The first principal component or eigenvector accounts for as much of the variability in the data as possible, and each succeeding component accounts for as much of the remaining variability as possible. The main objective of PCA is to reduce the dimensionality of the data set and to identify new underlying variables.

Principal component analysis compares the structure of two or more covariance matrices in a hierarchical fashion. For instance, one matrix might be identical to another except that each element of the matrix is multiplied by a single constant. The matrices are thus proportional to one another. More particularly, the matrices share identical eigenvectors (or principal components), but their eigenvalues differ by a constant. Another relationship between matrices is that they share principal components in common, but their eigenvalues differ. The mathematical technique used in principal component analysis is called eigenanalysis. The eigenvector associated with the largest eigenvalue has the same direction as the first principal component. The eigenvector associated with the second largest eigenvalue determines the direction of the second principal component. The sum of the eigenvalues equals the trace of the square matrix and the maximum number of eigenvectors equals the number of rows of this matrix.

In another embodiment, the algorithm is a classifier. One type of classifier is created by “training” the algorithm with data from the training set and whose performance is evaluated with the test set data. Examples of classifiers used in conjunction with the invention are discriminant analysis, decision tree analysis, receiver operator curves or split and score analysis.

The term “decision tree” refers to a classifier with a flow-chart-like tree structure employed for classification. Decision trees consist of repeated splits of a data set into subsets. Each split consists of a simple rule applied to one variable, e.g., “if value of “variable 1” larger than “threshold 1”; then go left, else go right”. Accordingly, the given feature space is partitioned into a set of rectangles with each rectangle assigned to one class.

The terms “test set” or “unknown” or “validation set” refer to a subset of the entire available data set consisting of those entries not included in the training set. Test data is applied to evaluate classifier performance. The terms “training set” or “known set” or “reference set” refer to a subset of the respective entire available data set. This subset is typically randomly selected, and is solely used for the purpose of classifier construction.

The following examples are presented in order to more fully illustrate some embodiments of the invention. They should, in no way be construed, however, as limiting the broad scope of the invention.

### EXAMPLES

### Materials and Methods

Human Subjects

The study was approved by the Institutional Review Boards of each participating clinical unit; informed consent was obtained from all participants. In an initial study, sera derived from blood samples obtained from 22 healthy subjects, 18 Pemphigus Vulgaris (PV) patients, 15 Scleroderma and Systemic Sclerosis (SSc) patients, and 34 Systemic Lupus Erythematosus (SLE) patients were tested using an antigen microarray that included A20, C20, G20 and T20 single-stranded oligonucleotides. In a follow-up study, sera samples obtained from 23 healthy subjects, 24 SSc patients, and 49 SLE patients were tested using an antigen microarray that included 58 single-stranded oligonucleotides. Overall, 60 SLE patients, 26 SSc patients, 18 PV patients, and 31 healthy subjects were tested. SLE and SSc patients were diagnosed according to clinically accepted criteria (Criteria published by E M Tan et al. Arthritis Rheum 1982; 25:1271, updated by MC Hochberg, Arthritis Rheum 1997; 40:1725; Preliminary criteria for the classification of systemic sclerosis (scleroderma). Subcommittee for scleroderma criteria of the American Rheumatism Association Diagnostic and Therapeutic Criteria Committee. Arthritis Rheum. 1980; 23(5):581-90). The diagnosis of PV was based upon clinical features and laboratory tests: suprabasal separation on histology of skin lesions, positive direct and indirect immunofluorescence microscopy, and/or ELISA detection of anti-desmoglein Abs (Zagorodniuk I, et al. Int J Dermatol. 2005 July; 44(7):541-4).

Blood samples and clinical data were collected from patients arriving at the Rheumatology and Nephrology Units at Rabin Medical Center, PetachTikva, Israel; the Rheumatology Unit and the Hematology Department of the Sheba Medical Center, Israel; the Department of Dermatology, Tel Aviv Sourasky Medical Center; and the Dipartimento di Scienze Mediche e Chirurgiche, Sezione di Clinica Medica, Polo Didattico, Ancona, Italy. Inclusion criteria were ACR criteria score of >3 at time of diagnosis. Healthy control samples were obtained under study protocols approved by the Institutional Review Boards of each participating clinical unit; informed consent was obtained from all participants.

Antigens and Serum Testing

In a follow-up study, 58 different oligonucleotides, as well as double and single stranded DNA, in various lengths (104 different preparations overall), were spotted on epoxy-activated glass substrates (ArrayIt SuperEpoxi microarray substrate slides, Sunnyvale, Calif.). The oligonucleotides were purchased from SBS Genetech Co., Ltd. (Shanghai, China). The microarrays were then blocked for 1 hour at 37° with 1% bovine serum albumin. Test serum samples in 1% Bovine Serum Albumin (BSA) blocking buffer (1:10 dilution) were incubated under a coverslip for 1 hour at 37°. The arrays were then washed and incubated for 1 hour at 37° with a 1:500 dilution of two detection antibodies, mixed together: a goat anti-human IgG Cy3-conjugated antibody, and a goat anti-human IgM Cy5-conjugated antibody (both purchased from Jackson ImmunoResearch Laboratories Inc., West Grove, Pa.). Image acquisition was performed by laser (Agilent Technologies, Santa Clara, Calif.) and the results were analyzed using Quantarray software (Packard BioChip Technologies, Billerica, Mass.). The quantitative range of signal intensity of binding to each antigen spot was 0-65,000; this range of detection made it possible to obtain reliable data at a 1:10 dilution of test serum samples.

Alternatively, oligonucleotides and double and single stranded DNA in various chain lengths were spotted on epoxyhexyltriethoxysilane (EHTES) activated slides. The microarrays were then blocked for 1 hour at room temperature with 1% casein. Test serum samples in 1% casein blocking buffer (1:20 dilution) were incubated under a coverslip for 1 hour at 37°. The arrays were then washed and incubated for 1 hour at 37° with a 1:500 dilution of two detection antibodies, mixed together: a goat anti-human IgG Cy3-conjugated antibody, and a goat anti-human IgM AF647-conjugated antibody (both purchased from Jackson ImmunoResearch Laboratories Inc., West Grove, Pa.). Image acquisition was performed by laser at two wavelengths 530 nm and 630 nm (Agilent Technologies, Santa Clara, Calif.) and the results were analyzed using Genepix Pro 7.0 software with default settings. The quantitative range of signal intensity of binding to each antigen spot was 0-65,000; this range of detection made it possible to obtain reliable data at a 1:20 dilution of test samples.

Image Analysis and Data Processing

Each spot's intensity is represented by its pixels' mean after subtraction of its local background median, followed by Log 2 transform. Negative spots (following background subtraction) are imputed with background-like intensity. The foreground and background intensities of multiple spots of each antigen were averaged, and the difference between the foreground and the background was calculated. The resulting value was taken as the antigen reactivity of the antibodies binding to that spotted antigen. All antigens showed meaningful reactivity in a significant number of slides; thus no antigen was excluded.

Statistical Analysis of Antibody Results

Antigens whose reactivity was higher or lower in a specific study subgroup compared to other subgroups were identified. Antigens that allowed for setting a classification threshold such as positive predictive value (PPV)≧90% and sensitivity≧20% were achieved and determined to significantly characterize a specific subgroup. SLE patients were marked positive for dsDNA if their reactivity to dsDNA passed this requirement. For added restriction, only antigens whose p value for a two sided t-test (after Benjamini-Hochberg correction for multiple hypothesis) was smaller than 0.05 were selected.

### Example 1

### Antibody Binding to Homo-Nucleotide 20-Mers

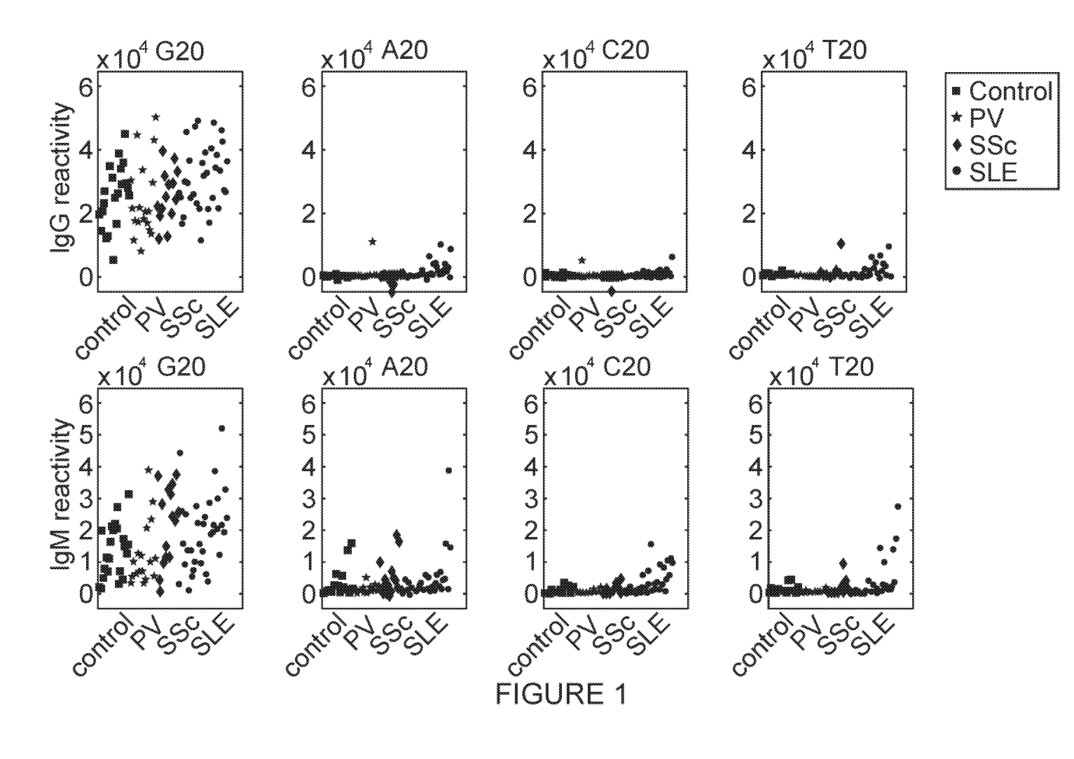

Sera samples from healthy subjects, PV, SSc and SLE patients were tested for binding of serum IgG and IgM antibodies to four 20-mer homo-nucleotides: G20 (SEQ ID NO: 43), A20 (SEQ ID NO: 22), C20 (SEQ ID NO: 15), and T20 (SEQ ID NO: 8) (FIG. 1). The reactivities were ordered by each subject's reactivity to dsDNA, from left to right. It can be seen that IgG reactivities to G20 (SEQ ID NO: 43) were very high in all subjects, and significantly higher than the very low reactivities to the other oligonucleotides. However, PV patients were found to have significantly lower IgG and IgM reactivities to G20 than did SSc patients. Apart from that, no difference was found between the study groups.

IgG reactivities to A20, C20 and T20 in SLE patients correlated with their reactivities to dsDNA; Patients with low reactivities to dsDNA did not manifest reactivities to A20, C20 and T20, but several patients with higher reactivities to dsDNA showed some reactivities to A20, C20 and T20 (FIG. 1). Healthy subjects, SSc and PV patients had very low or no reactivities to A20, C20 and T20.

IgM reactivities to the four homo-nucleotide sequences were more diffuse: some subjects in each group showed high reactivities to G20, but, in contrast to the IgG reactivities to G20, some of the sera showed little or no IgM binding to G20.

To further characterize the antibodies' reactivity against poly-G and poly-T oligonucleotides, an additional study on 23 healthy subjects, 24 SSc patients, and 49 SLE patients was performed. An extended microarray antigen chip was used. The chip contained 58 oligonucleotides, including poly-G and poly-T sequences with and without modifications (see below). The SLE patients were divided according to their reactivity to dsDNA in order to see if dsDNA positivity or negativity was associated with antibodies to the synthetic oligonucleotides. The results of the IgM and IgG reactivities to G20 (SEQ ID NO: 43), A20 (SEQ ID NO: 22), C20 (SEQ ID NO: 15), and T20 (SEQ ID NO: 8) of the first study were confirmed. Combining both studies yielded a total of 60 SLE patients, 26 SSc patients, 18 PV patients and 31 healthy subjects who all displayed high IgG reactivities to G20 (SEQ ID NO: 43) and relatively low reactivities to A20 (SEQ ID NO: 22), C20 (SEQ ID NO: 15), and T20 (SEQ ID NO: 8). Mean IgG reactivities to G20 (SEQ ID NO: 43) where significantly higher than the other oligonucleotides in all the study groups.

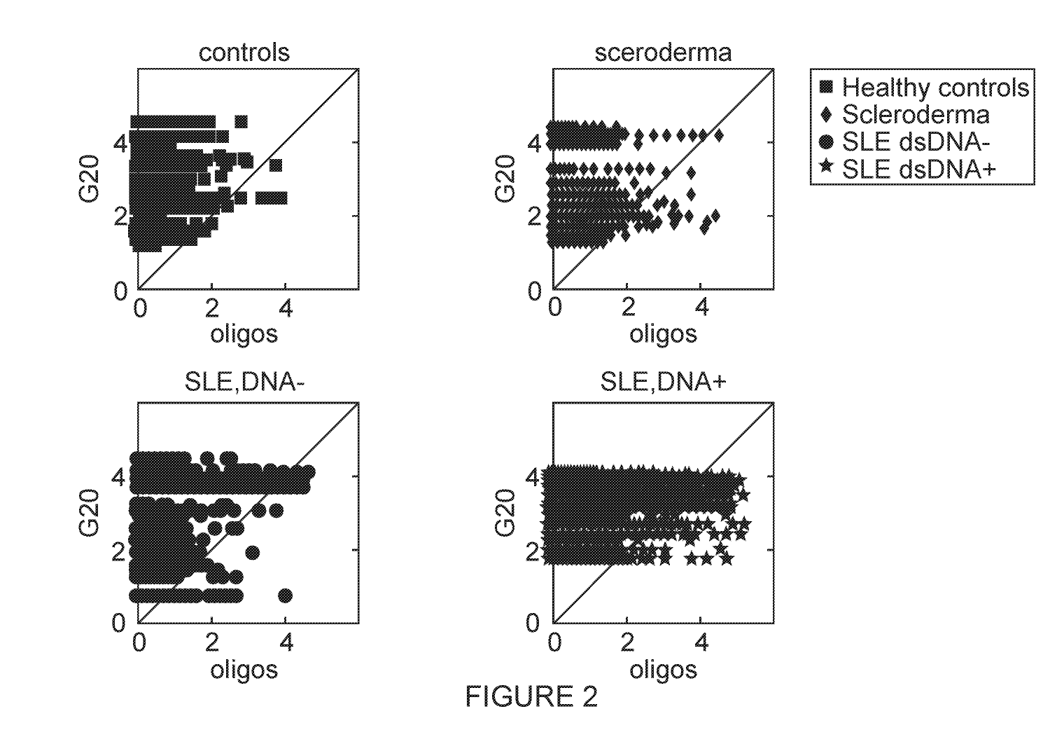

The IgG reactivities to G20 (SEQ ID NO: 43) were compared to IgG reactivities to the other oligonucleotides and to ssDNA, and dsDNA. FIG. 2 shows a scatter plot in which each dot represents the IgG reactivity to G20 (SEQ ID NO: 43) divided by a specific oligonucleotide. Since some of the oligonucleotides and ssDNA and dsDNA were in replicates and mixtures, each subject is represented by 97 spots. Higher reactivity to G20 (SEQ ID NO: 43) compared to a different oligonucleotide would be represented by a dot above the diagonal. Note that out of the 2231 spots of the 23 healthy subjects, only 10 were below the diagonal. This number increases a little for SSc patients and dsDNA-negative SLE patients, and peaks for dsDNA-positive patients (FIG. 2). Nevertheless, the average IgG reactivities to G20 (SEQ ID NO: 43) were significantly higher than IgG reactivities to any other oligonucleotide in all four subgroups tested.

The list of oligonucleotides tested on the antigen chip was as follows: A20 (SEQ ID NO: 22), C20 (SEQ ID NO: 15), T2G16T2 (SEQ ID NO: 10), G2T16G2 (SEQ ID NO: 16), (GA)10 (SEQ ID NO: 44), (GT)10 (SEQ ID NO: 45), G4-7,9,11,14,17,20 (SEQ ID NOs: 46, 37, 13, 17, 34, 47, 41, 36 and 43, respectively), T4-7,9,11,14,17,20 (SEQ ID NOs: 48, 49, 35, 9, 40, 4, 2 and 8, respectively), (CG)2-6,8,10 (SEQ ID NOs: 51, 52, 53,54, 29, 55 and 25, respectively), (C*G)2-6,8,10 (*=with C methyl) (SEQ ID NOs: 56, 57, 39, 58, 30, 33 and 26, respectively), T1G16T1 (SEQ ID NO: 20), G16T1 (SEQ ID NO: 18), T1G16 (SEQ ID NO: 38), G16T2 (SEQ ID NO: 12), T2G16 (SEQ ID NO: 24), G1T16G1 (SEQ ID NO: 5), T16G1 (SEQ ID NO: 7), G1T16 (SEQ ID NO: 42), T16G2 (SEQ ID NO: 28), G2T16 (SEQ ID NO: 14), GACGCT (SEQ ID NO: 59), GACGTT (SEQ ID NO: 6), G10GACGCT (SEQ ID NO: 27), G10GACGTT (SEQ ID NO: 60), GAGCCT (SEQ ID NO: 21), GAGCTT (SEQ ID NO: 61), G10GAGCCT (SEQ ID NO: 19), G10GAGCTT (SEQ ID NO: 62), CCCGGA (SEQ ID NO: 63), G10CCCGGA (SEQ ID NO: 32), and TCCATAACGTTGCAACGTTCTG (SEQ ID NO: 64).

### Example 2

### Antibody Binding to Poly-G or Poly-T is Related to the Length of the Homo-Nucleotide

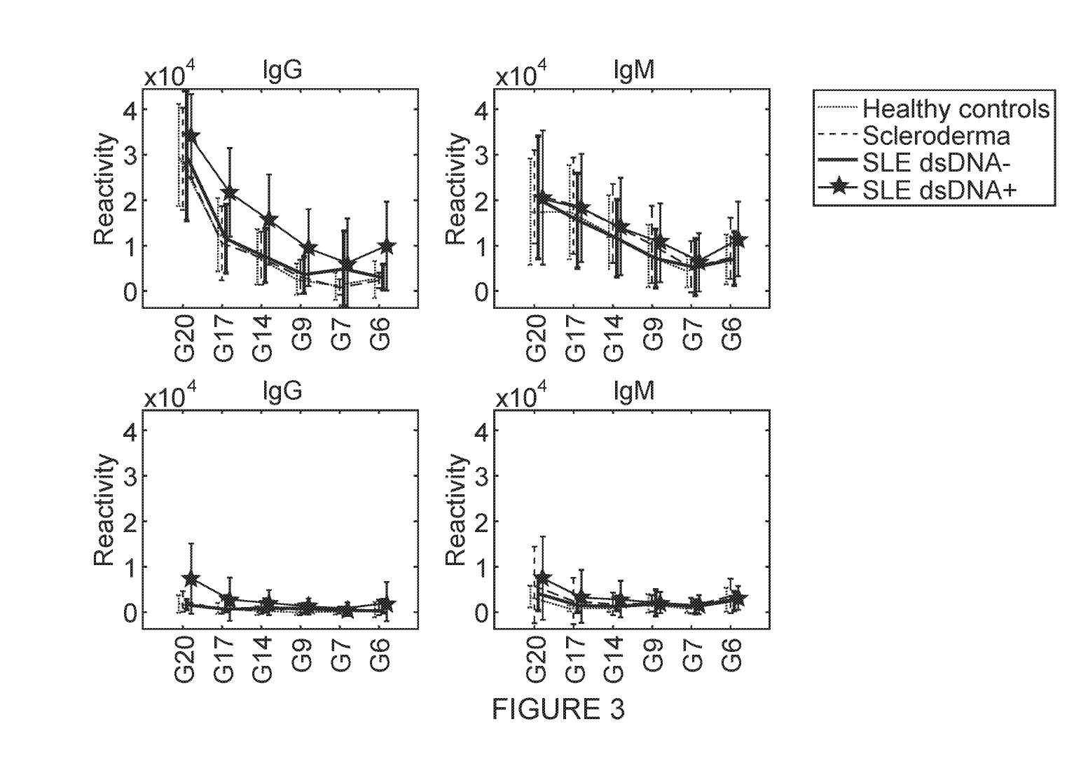

FIG. 3 shows the effect of variable lengths of the nucleotide oligomers on the mean IgG and IgM binding of each tested group to the T or G homo-nucleotides. It can be seen that, except for SLE patients positive for anti-dsDNA who showed reactivities to T20, none of the other groups showed appreciable IgG or IgM mean reactivities to any of the poly-T homo-nucleotides. In contrast, mean IgG reactivities to poly-G in all of the sera were high to G20 (SEQ ID NO: 43) and fell significantly as the lengths of the nucleotide chains were reduced to G17 (SEQ ID NO: 36) and below. Surprisingly, SLE patients positive for anti-dsDNA manifested higher mean IgG reactivities to the shorter G polymers than did the other groups.

Mean IgM binding to G20 (SEQ ID NO: 43) was lower than the IgG binding, and IgM binding was also affected by shortening the length of the oligomer. Note that the mean IgM binding of the SLE patients positive to dsDNA did not differ from that of the other groups.

### Example 3

### The Effects of Adding a Single T to Either the 5′ or the 3′ Termini of G16

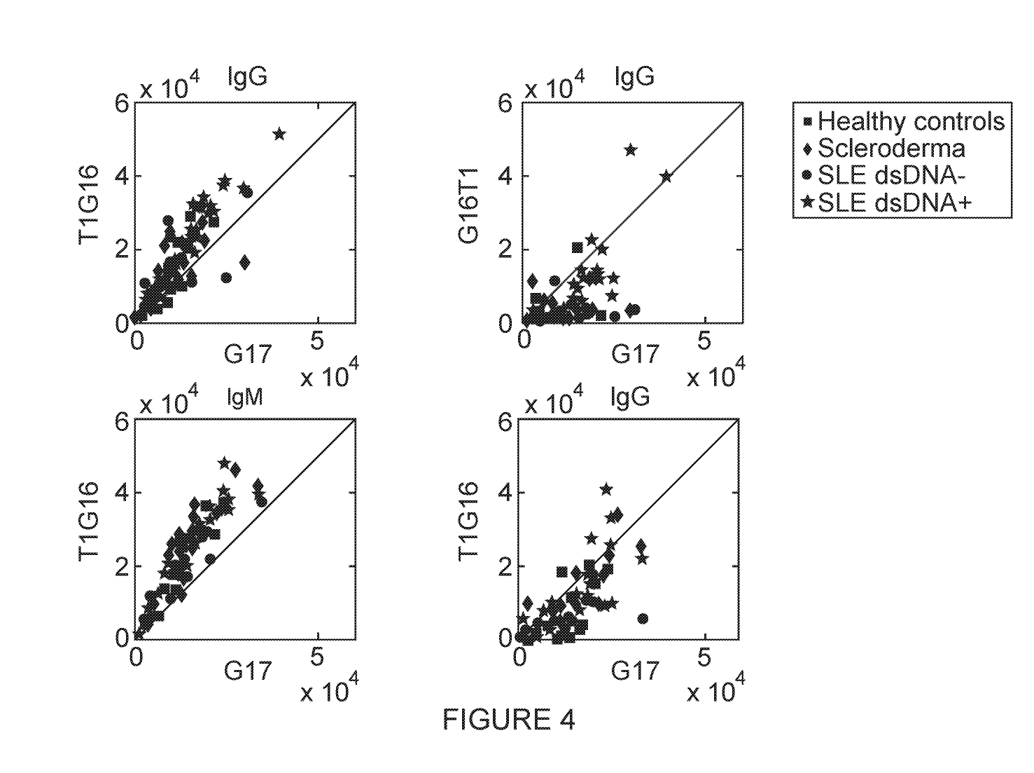

The degree of binding of IgG or IgM to G17 (SEQ ID NO: 36) compared to G16 to which a single T had been added either at the 5′ or 3′ end of the G-oligonucleotide chain was tested. FIG. 4 shows the results for individual subjects. It can be seen that both the IgG and IgM binding to T1G16 (SEQ ID NO: 38) was essentially equal to the binding to the G17 (SEQ ID NO: 36) chain, as evident from the diagonal between G17 (SEQ ID NO: 36) and T1G16 (SEQ ID NO: 38). However, the binding of each subject to G16T1 (SEQ ID NO: 18) was considerably less than the binding to G17 (SEQ ID NO: 36); a diagonal relationship was no longer present. Thus, it would appear that the reactivities to poly-G in each of the subject groups was highly influenced by the addition of a single T moiety to the 3′ end of the poly-G chain but not by the addition of a T to the 5′ end of the G chain; the spatial order of the nucleotides would appear to form an antigen structure critical to antibody binding.

### Example 4

### The Effects of Single G Additions to the Ends of Poly-T Sequences

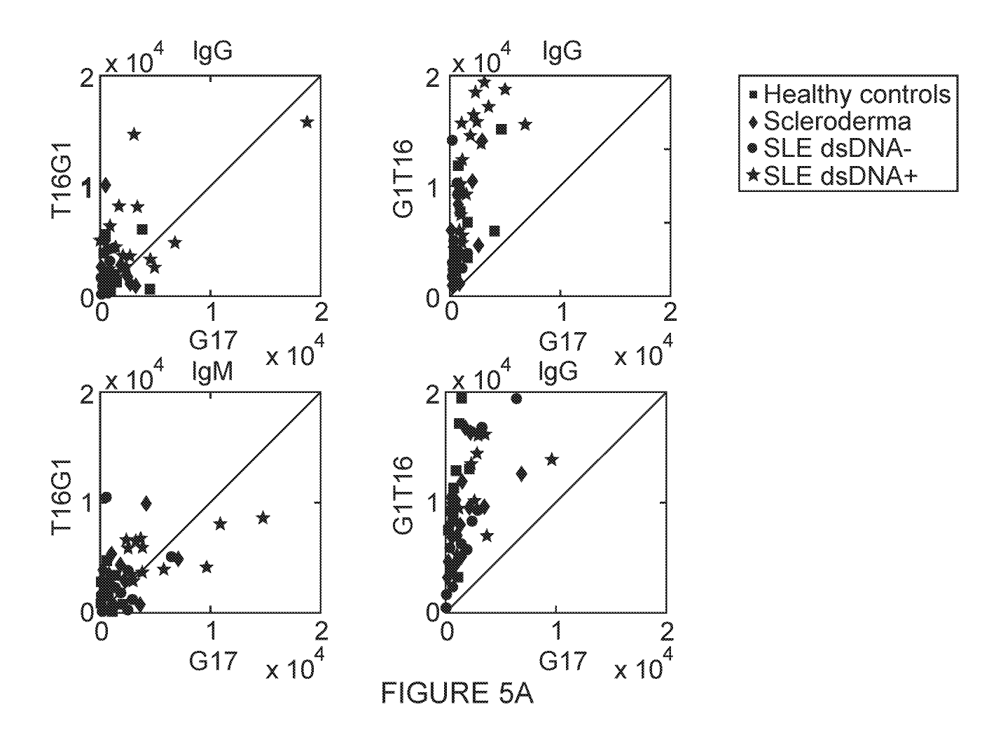

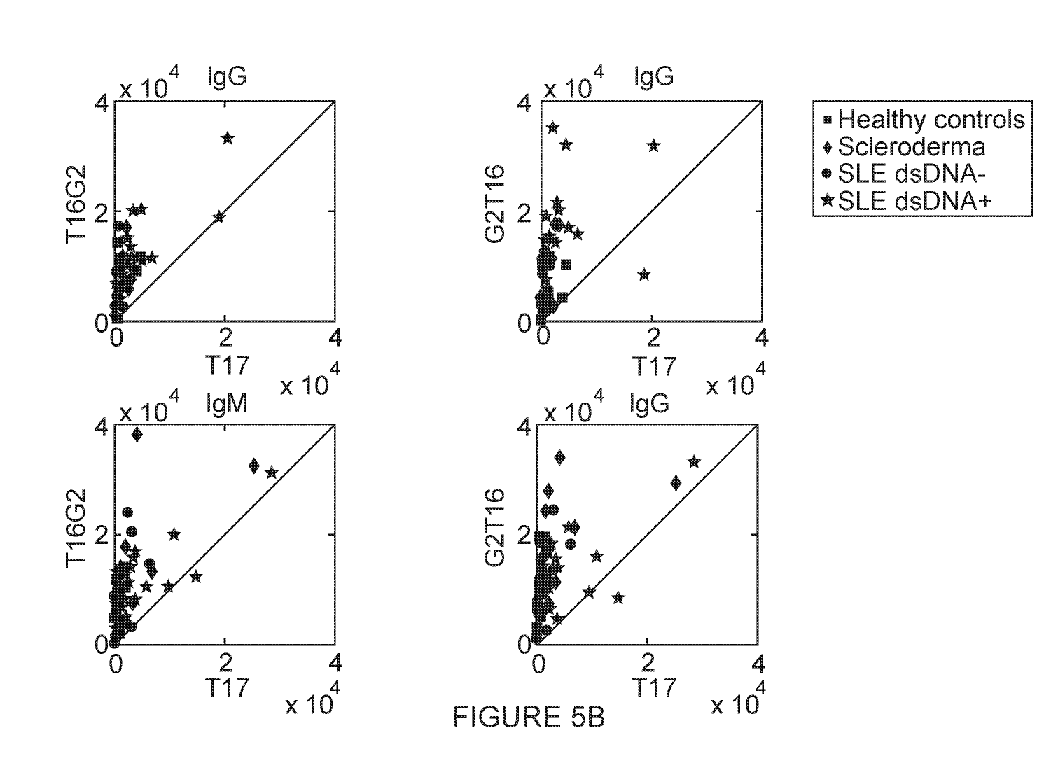

In view of the marked effects of adding a single T to the 3′ end of a poly-G chain (Example 3), the effects on IgG or IgM antibody binding by adding a single G to either the 5′ or 3′ end of a poly-T chain was tested. FIG. 5A shows that although both IgM and IgG reactivities to G1T16 (SEQ ID NO: 42) and to T16G1 (SEQ ID NO: 7) increased in almost all the subjects (most points are above the diagonal), the increases were much more pronounced when the guanine was added to the 5′ end. Similarly IgM and IgG reactivities to G2T16 (SEQ ID NO: 14) and T16G2 (SEQ ID NO: 28) were also increased compared to T17 (SEQ ID NO: 2) (FIG. 5B).

In summary, reactivities to poly-T oligonucleotides could be increased significantly by the addition of even a single G to either end of the chain; this was in marked contrast to the inhibition of antibody binding to poly-G by the addition of a single T to the 3′ end of the chain.

### Example 5

### Reactivities to CpG Repeats

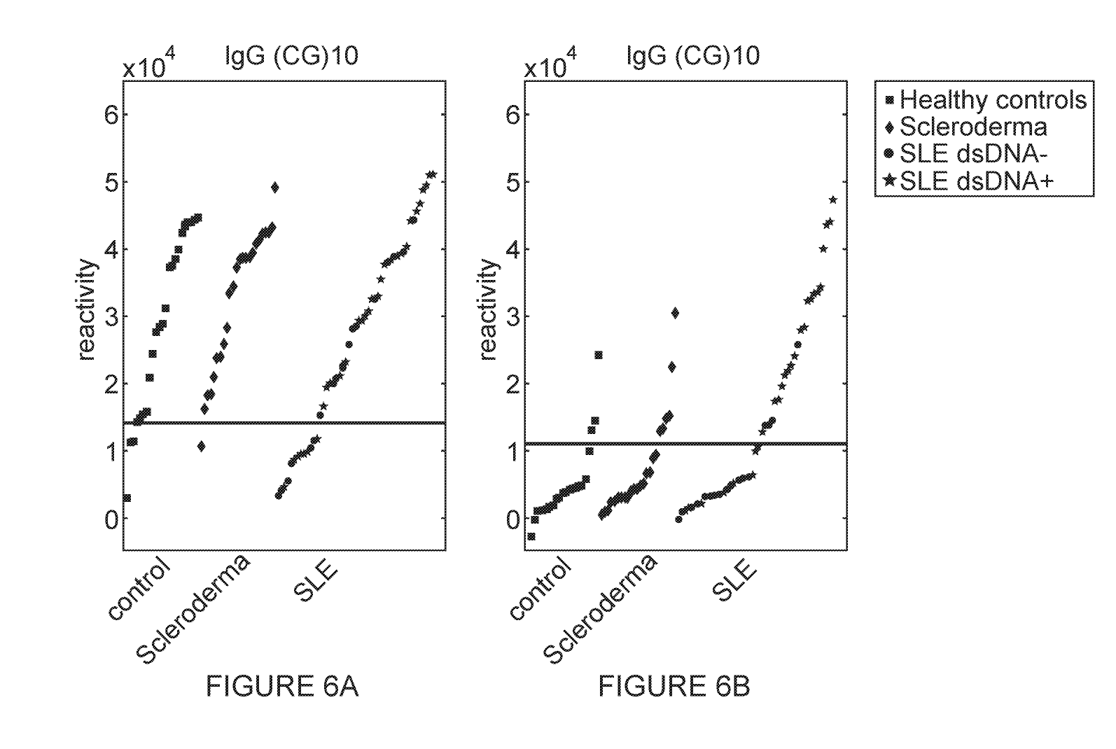

IgM reactivities were measured in three subgroups to a 20-mer formed by 10 repetitions of the C-G di-nucleotides, (CG)10 (SEQ ID NO: 25). IgM reactivities to (CG)10 (SEQ ID NO: 25) were high in all but one of the healthy subjects, in all of the SSc patients and in most of the SLE patients. Indeed, a subgroup of SLE patients manifested low IgM reactivities to (CG)10 (SEQ ID NO: 25), a significant difference from the SSc patients (FIG. 6). A group of SLE patients, mainly those positive for anti-dsDNA, manifested high IgG reactivities to (CG)10 (SEQ ID NO: 25).

### Example 6

Poly-G Autoantibody Levels are Decreased in Transplant Patients Receiving Immunosuppression Drugs

In order to verify that poly-G autoantibody levels are decreased in subjects with a suppressed immunological system, a study was designed to examine poly-G autoantibody levels in kidney transplanted patients 6 months after administrating of two different immunosuppression drugs, Cyclosporine A (CsA, Trade names Neoral and Sandimmune) or belatacept (Trade name Nulojix).

The study included the following antigens: G14 (SEQ ID NO: 41), G17 (SEQ ID NO: 36), G20 (SEQ ID NO: 43), G30 (SEQ ID NO: 65) and G40 (SEQ ID NO: 66). Each oligonucleotide was diluted using 7 different diluents in order to ensure the printing performance of each antigen.

Printed slides were hybridized with the following serum samples: samples collected from 6 kidney transplanted patients administrated with belatacept at baseline and 6 months post transplant; samples collected from 6 kidney transplanted patients administrated with CsA at baseline and 6 months post transplant; and samples collected from 6 healthy individuals.

Belatacept dosing was 10 mg/kg on days 1 and 5, then on weeks 2, 4, 6, 8, 10 and 12, 10 mg/kg monthly at month 4, 5 and 6, and then 5 mg/kg monthly thereafter. CyA dosing was 4-10 mg/kg, adjusted to maintain blood level (by standard TDM) of 150-300 ng/ml, 0-30 days, then at 100-200 ng/ml thereafter. All patients received basiliximab (IL-2 receptor MAb blocking antibody), mycophenolate mofetil 2 g/day, divided into two doses 12 hours apart, and corticosteroids, 500 mg IV Day 0, tapered to 2.5 mg po by Day 15.

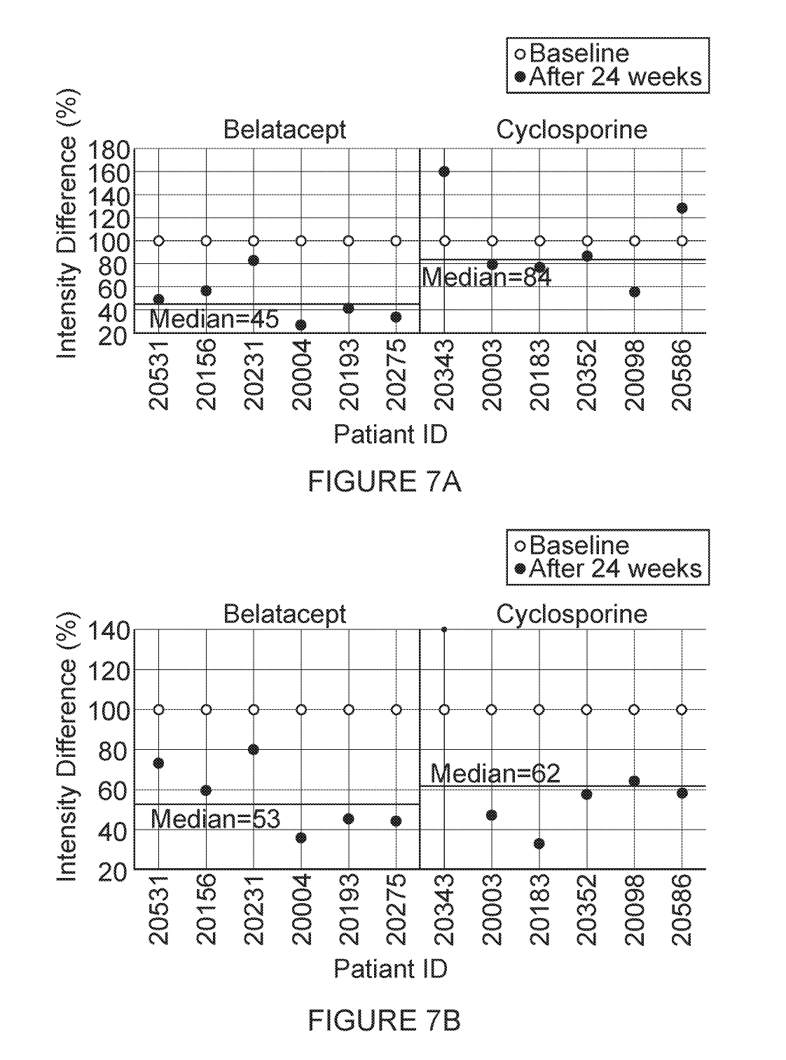

FIGS. 7 to 12 provide a detailed analysis of antibody binding intensity (%) in belatacept- and CsA-treated patients at baseline and 24 weeks after transplantation (Tx), for each antigen. The data is summarized in Table 1.

TABLE 1
The effect of belatacept and CsA on antibody levels against
poly-G antigens.

| | Antibody level 24 weeks after | Antibody level 24 weeks |
|---|---|---|
| | transplantation and | after transplantation |
| | belatacept administration | and CsA administration |
| | compared to baseline | compared to baseline |
| | (%, median intensity) | (%, median intensity) |
| IgM levels | 45 | 84 |
| against G14 | | |
| (SEQ ID NO: | | |
| 41) | | |
| IgG levels | 53 | 62 |
| against G14 | | |
| (SEQ ID NO: | | |
| 41) | | |
| IgM levels | 63 | 94 |
| against G17 | | |
| (SEQ ID NO: | | |
| 36) | | |
| IgG levels | 70 | 76 |
| against G17 | | |
| (SEQ ID NO: | | |
| 36) | | |
| IgM levels | 63 | 77 |
| against G20 | | |
| (SEQ ID NO: | | |
| 43) | | |
| IgG levels | 58 | 78 |
| against G20 | | |
| (SEQ ID NO: | | |
| 43) | | |
| IgM levels | 59 | 73 |
| against G30 | | |
| (SEQ ID NO: | | |
| 65) | | |
| IgG levels | 67 | 108 |
| against G30 | | |
| (SEQ ID NO: | | |
| 65) | | |
| IgM levels | 72 | 91 |
| against G40 | | |
| (SEQ ID NO: | | |
| 66) | | |
| IgG levels | 81 | 53 |
| against G40 | | |
| (SEQ ID NO: | | |
| 66) | | |

G14 IgM (FIG. 7A)

The median intensity of sera from subjects treated with belatacept 24 weeks after Tx was 45% of baseline intensity. There was a reduction of 55% in G14 IgM autoantibody intensity. This reduction was observed in all tested patients. The median intensity of sera obtained from subjects treated with CsA 24 weeks after Tx was 84% of baseline intensity. There is a reduction of 16%.

G14 IgG (FIG. 7B)

The median intensity of sera from subjects treated with belatacept 24 weeks after Tx is 53% of baseline intensity. There is a reduction of 47% in G14 IgG autoantibody intensity. This reduction was observed in all tested patients. The median intensity of sera obtained from subjects treated with CsA 24 weeks after Tx is 62% of baseline intensity. There is a reduction of 38%.

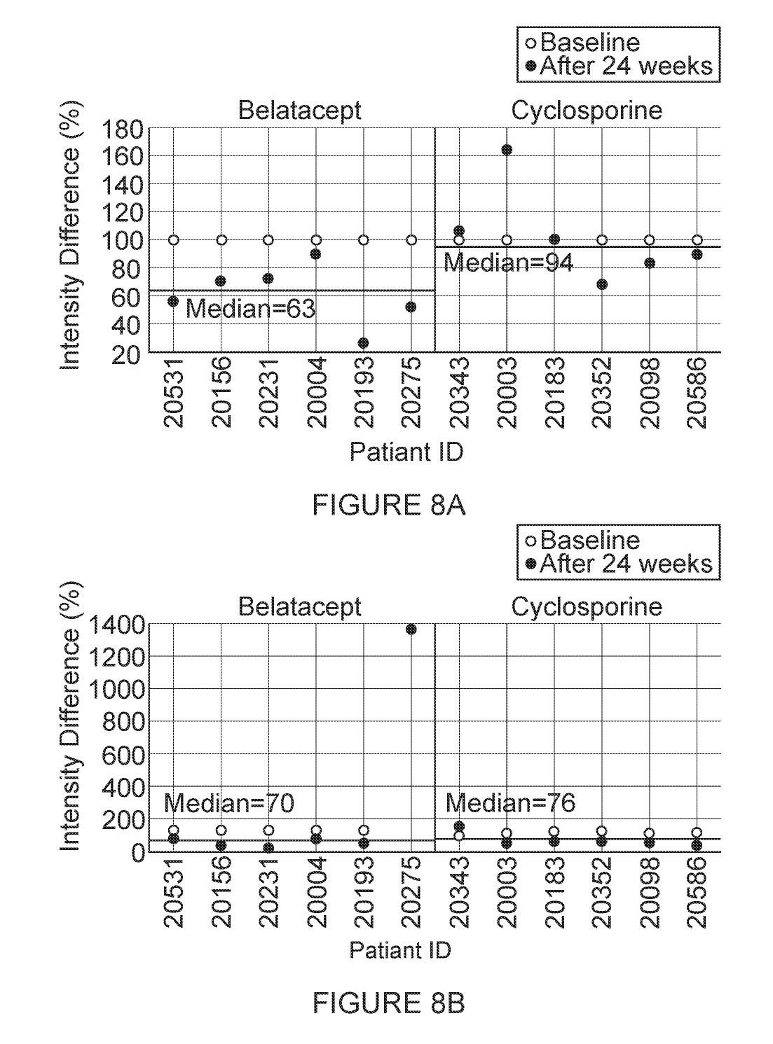

G17 IgM (FIG. 8A)

The median intensity of sera obtained from subjects treated with belatacept 24 weeks after Tx is 63% of baseline intensity. There is a reduction of 37% in G17 IgG autoantibody intensity. This reduction was observed in all tested patients. The median intensity median of sera obtained from subjects treated with CsA 24 weeks after Tx is 94% of baseline intensity. There is a reduction of 6%.

G17 IgG (FIG. 8B)

The median intensity of sera obtained from subjects treated with belatacept 24 weeks after Tx is 70% of baseline intensity. There is a reduction of 30% in G17 IgG autoantibody intensity. This reduction was observed in all tested patients. The median intensity of sera obtained from subjects treated with CsA 24 weeks after Tx is 76% of baseline intensity. There is a reduction of 24%.

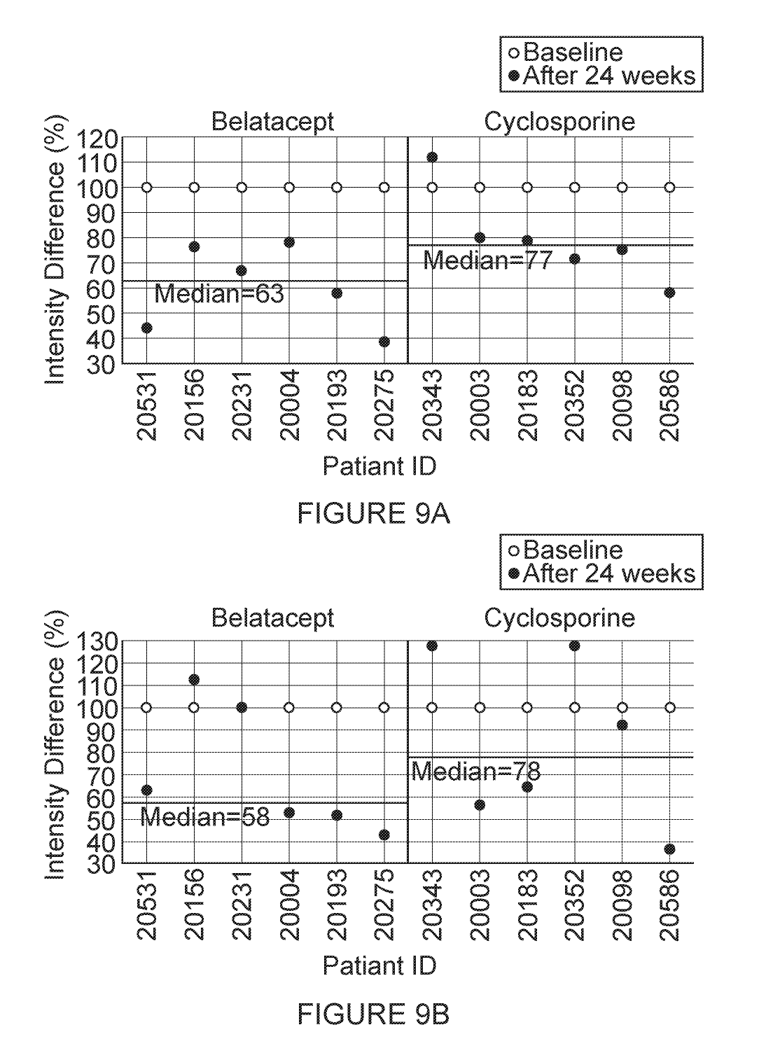

G20 IgM (FIG. 9A)

The median intensity of sera obtained from subjects treated with belatacept 24 weeks after Tx is 63% of baseline intensity. There is a reduction of 37% in G20 IgM autoantibody intensity. This reduction was observed in all patients. The median intensity of sera obtained from subjects treated with CsA 24 weeks after Tx is 77% of baseline intensity. There is a reduction of 23%.

G20 IgG (FIG. 9B)

The median intensity of sera obtained from subjects treated with belatacept 24 weeks after Tx is 58% of baseline intensity. There is a reduction of 42% in G20 IgG autoantibody intensity. The median intensity of sera obtained from subjects treated with CsA 24 weeks after Tx is 78% of baseline intensity. There is a reduction of 22%.

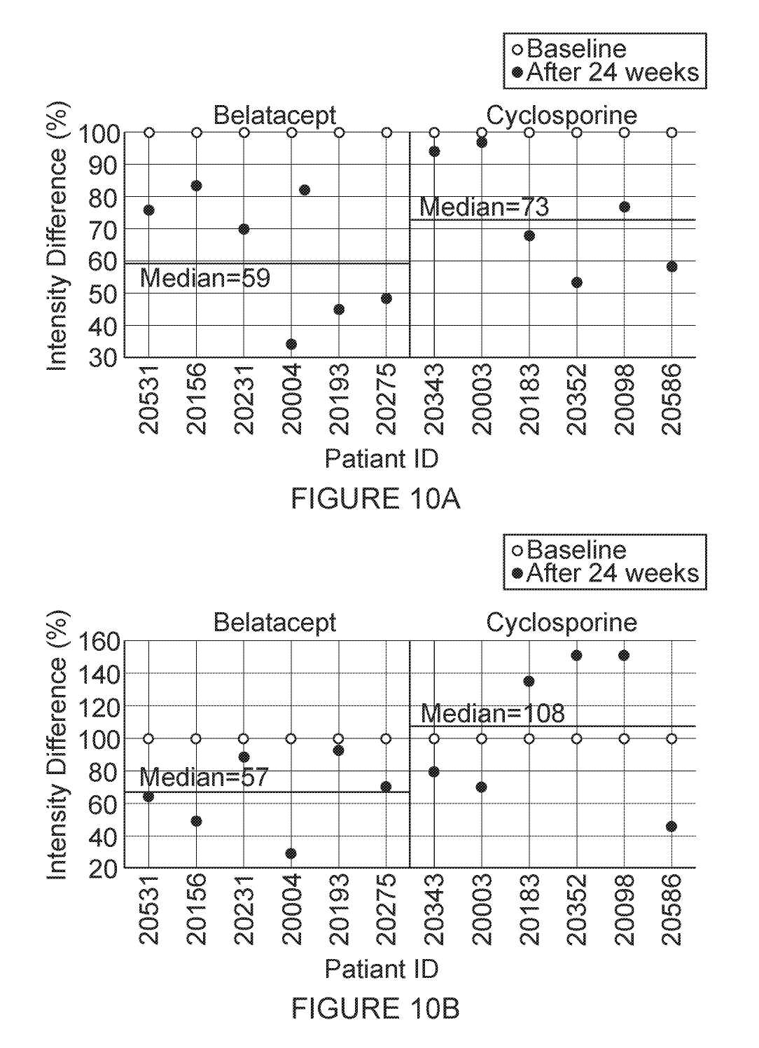

G30 IgM (FIG. 10A)

The median intensity of sera obtained from subjects treated with belatacept 24 weeks after Tx is 59% of baseline intensity. There is a reduction of 41% in G30 IgM autoantibody intensity. This reduction was observed in all tested patients. The median intensity of sera obtained from subjects treated with CsA 24 weeks after Tx is 73% of baseline intensity. There is a reduction of 27%. This reduction was observed in all tested patients.

G30 IgG (FIG. 10B)

The median intensity of sera obtained from subjects treated with belatacept 24 weeks after Tx is 67% of baseline intensity. There is a reduction of 33% in G30 IgG autoantibody intensity. This reduction was observed in all tested patients. The median intensity of sera obtained from subjects treated with CsA 24 weeks after Tx is 108% of baseline intensity. There is an increase of 8%.

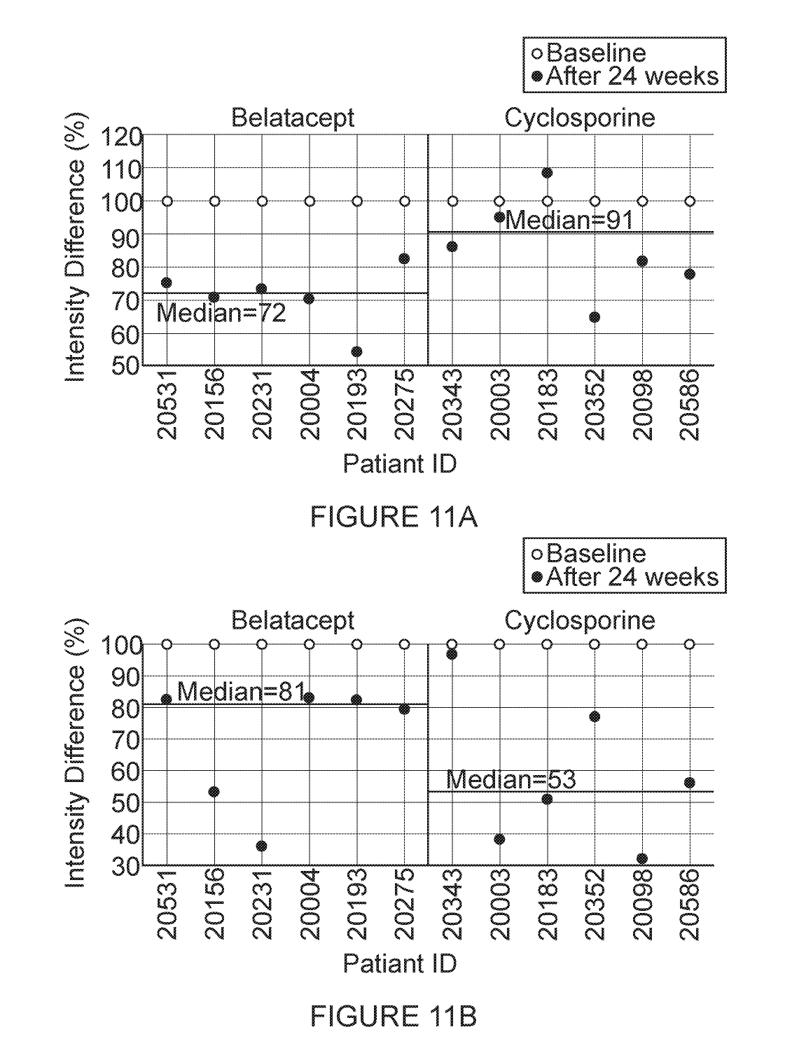

G40 IgM (FIG. 11A)

The median intensity of sera obtained from subjects treated with belatacept 24 weeks after Tx is 72% of baseline intensity. There is a reduction of 28% in G40 IgM autoantibody intensity. This reduction was observed in all tested patients. The median intensity of sera obtained from subjects treated with CsA 24 weeks after Tx is 91% of baseline intensity. There is a reduction of 9%.

G40 IgG (FIG. 11B)

The median intensity of sera obtained from subjects treated with belatacept 24 weeks after Tx is 81% of baseline intensity. There is a reduction of 19% in G40 IgG autoantibody intensity. This reduction was observed in all tested patients. The median intensity of sera obtained from subjects treated with CsA 24 weeks after Tx is 53% of baseline intensity. There is a reduction of 47%. This reduction was observed in all patients.

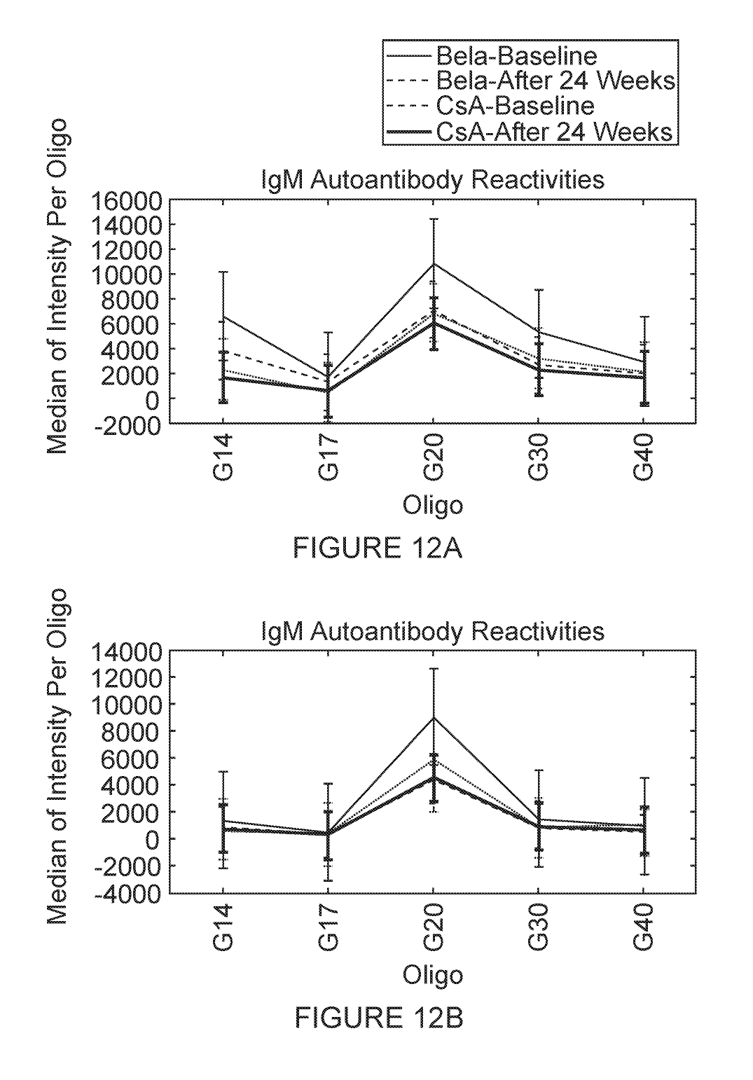

Examining the combined poly-G reactivity, as demonstrated in FIGS. 12A-B, a greater reduction in autoantibodies median intensity was observed in belatacept-treated patients than in CsA-treated patients. This median intensity reduction was greater in the IgM panel (FIG. 12A) than in the IgG panel (FIG. 12B). In addition, a significant reduction was observed mainly in the G20 autoantibody intensity.

Example 6 demonstrates a reduction in poly-G autoantibody intensity, observed 6 months post immunosuppression administration in patients treated with belatacept or CsA. This reduction was observed in all belatacept patients and in almost all of the CsA tested patients. A clear reduction was observed in belatacept patients mainly in the IgM isotype. A greater reduction was observed in G14 autoantibodies when compared to other oligonucleotides. Although the number of tested patients was restricted to 6 in each group, the reduction pattern is clear.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention.

1-32. (canceled)

33. A method of determining a level of immunological competence of a subject, the method comprising:
(i) obtaining a sample from the subject;
(ii) assaying the sample for the presence of antibodies to at least one oligonucleotide antigen comprising at least 10 contiguous guanine nucleotides, thereby determining the reactivity of the antibodies in the sample to the at least one oligonucleotide antigen; and
(iii) comparing the antibody reactivity to the reactivity of a control subject or a reference control value;
wherein an equal or higher reactivity of the antibodies in the sample obtained from the subject compared to the reactivity of the control subject is an indication that the subject has a competent immunological system, and wherein a significantly lower reactivity of the antibodies in the sample obtained from the subject compared to control is an indication that the subject has an incompetent immunological system.

34. The method of claim 33, wherein the subject is selected from the group consisting of a transplant patient, an organ recipient, a subject being evaluated as an organ recipient candidate, an immunocompromised subject, a subject which is or has been in a state of immune deficiency, a subject which is or has been in a state of malnutrition, a subject of a certain genetic profile associated with immunological-incompetence, a subject of a certain familial history associated with a state of immunological-incompetence, and a subject of a certain psychiatric state or condition associated with immunological-incompetence.

35. The method of claim 33, wherein the subject is or was receiving at least one immunosuppressive drug or an immunosuppressive treatment.

36. The method of claim 35, wherein the at least one immunosuppressive treatment is irradiation, or wherein the at least one immunosuppressive drug is selected from the group consisting of: calcineurin inhibitors (CNIs), T-cell costimulatory blockers, purine metabolism inhibitors, mTOR inhibitors, anti-lymphocyte globulins (ALGs), anti-thymocyte globulin (ATGs), monoclonal antibodies (mAbs) to OKT3, mAbs to IL-2 receptor, corticosteroids, and any combination thereof.

37. The method of claim 36, wherein the at least one immunosuppressive drug is a T-cell costimulatory blocker or a calcineurin inhibitor.

38. The method of claim 37, wherein the T-cell [symbol] costimulatory blocker is belatacept, or wherein the calcineurin inhibitor is Cyclosporine A.

39. The method of claim 33, wherein the reactivity of antibodies is selected from IgG reactivities and IgM reactivities.

40. The method of claim 33, wherein the at least one antigen is selected from the group consisting of G14 having the nucleotide sequence as set forth in SEQ ID NO: 41, G20 having the nucleotide sequence as set forth in SEQ ID NO: 43, G40 having the nucleotide sequence as set forth in SEQ ID NO: 66, G17 having the nucleotide sequence as set forth in SEQ ID NO: 36, and G30 having the nucleotide sequence as set forth in SEQ ID NO: 65.

41. The method of claim 40, wherein the at least one antigen is G14 having the nucleotide sequence as set forth in SEQ ID NO: 41, or wherein the at least one antigen is G20 having the nucleotide sequence as set forth in SEQ ID NO: 43.

42. The method of claim 33, comprising determining the reactivity of antibodies in the sample obtained from the subject to at least two different antigens selected from the group consisting of G14 having the nucleotide sequence as set forth in SEQ ID NO: 41, G20 having the nucleotide sequence as set forth in SEQ ID NO: 43, G40 having the nucleotide sequence as set forth in SEQ ID NO: 66, G17 having the nucleotide sequence as set forth in SEQ ID NO: 36, and G30 having the nucleotide sequence as set forth in SEQ ID NO: 65.

43. The method of claim 33, wherein the sample is selected from the group consisting of: serum, plasma and blood.

44. The method of claim 33, wherein the oligonucleotide sequence does not comprise one or more thymine at the 3′ terminus of the oligonucleotide sequence.

45. The method of claim 33, wherein the at least one antigen is used in the form of an antigen probe set.

46. The method of claim 33, further comprising a step selected from the group consisting of:
a) determining that a subject who has a competent immunological system is not amenable for organ transplantation;
b) determining that a subject who has a competent immunological system and receives immunosuppressive treatment or an immunosuppressive drug is amenable for higher dosages of the immunosuppressive treatment or the immunosuppressive drug than previously administered;
c) determining that a subject who has a suppressed or an incompetent immunological system is amenable for organ transplantation; and
d) determining that a subject who has a suppressed or an incompetent immunological system and receiving immunosuppressive treatment or an immunosuppressive drug is amenable for lower dosages of the immunosuppressive treatment or the immunosuppressive drug than previously administered.

47. The method of claim 46, further comprising administering higher dosages of the immunosuppressive treatment or the immunosuppressive drug than previously administered to the subject determined not amenable for organ transplantation or determined amenable for higher dosages of the immunosuppressive treatment or the immunosuppressive drug than previously administered.

48. The method of claim 46, further comprising administering lower dosages of the immunosuppressive treatment or the immunosuppressive drug than previously administered to the subject determined amenable for organ transplantation or determined to be amenable for lower dosages of the immunosuppressive treatment or the immunosuppressive drug than previously administered.

49. An antigen probe set comprising a plurality of different oligonucleotide antigens, each oligonucleotide antigen comprising at least 10 contiguous guanine nucleotides.

50. The antigen probe set of claim 49, comprising a plurality of different oligonucleotide antigens selected from the group consisting of G14 having the nucleotide sequence as set forth in SEQ ID NO: 41, G20 having the nucleotide sequence as set forth in SEQ ID NO: 43, G40 having the nucleotide sequence as set forth in SEQ ID NO: 66, G17 having the nucleotide sequence as set forth in SEQ ID NO: 36, and G30 having the nucleotide sequence as set forth in SEQ ID NO: 65.

51. An article of manufacture comprising the antigen probe set of claim 49, in the form of an antigen probe array or an antigen chip, or in the form of a kit, further comprising means for determining the reactivity of an antibody to an antigen.


SEQUENCE LISTING (67 sequences)

SEQ ID NO: 1 - DNA, 20 bp, Artificial
  Synthetic oligonucleotide
ccataattgcaaacgttctg

SEQ ID NO: 2 - DNA, 17 bp, Artificial
  Synthetic oligonucleotide
ttttttttttttttttt

SEQ ID NO: 3 - DNA, 20 bp, Artificial
  Synthetic oligonucleotide
ccataattgcaaagcttctg

SEQ ID NO: 4 - DNA, 14 bp, Artificial
  Synthetic oligonucleotide
tttttttttttttt

SEQ ID NO: 5 - DNA, 18 bp, Artificial
  Synthetic oligonucleotide
gttttttttttttttttg

SEQ ID NO: 6 - DNA, 6 bp, Artificial
  Synthetic oligonucleotide
gacgtt

SEQ ID NO: 7 - DNA, 17 bp, Artificial
  Synthetic oligonucleotide
ttttttttttttttttg

SEQ ID NO: 8 - DNA, 20 bp, Artificial
  Synthetic oligonucleotide
tttttttttttttttttttt

SEQ ID NO: 9 - DNA, 7 bp, Artificial
  Synthetic oligonucleotide
ttttttt

SEQ ID NO: 10 - DNA, 20 bp, Artificial
  Synthetic oligonucleotide
ttggggggggggggggggtt

SEQ ID NO: 11 - DNA, 20 bp, Artificial
  Synthetic oligonucleotide
ggggggggggtttttttttt

SEQ ID NO: 12 - DNA, 18 bp, Artificial
  Synthetic oligonucleotide
ggggggggggggggggtt

SEQ ID NO: 13 - DNA, 6 bp, Artificial
  Synthetic oligonucleotide
gggggg

SEQ ID NO: 14 - DNA, 18 bp, Artificial
  Synthetic oligonucleotide
ggtttttttttttttttt

SEQ ID NO: 15 - DNA, 20 bp, Artificial
  Synthetic oligonucleotide
cccccccccccccccccccc

SEQ ID NO: 16 - DNA, 20 bp, Artificial
  Synthetic oligonucleotide
ggttttttttttttttttgg

SEQ ID NO: 17 - DNA, 7 bp, Artificial
  Synthetic oligonucleotide
ggggggg

SEQ ID NO: 18 - DNA, 17 bp, Artificial
  Synthetic oligonucleotide
ggggggggggggggggt

SEQ ID NO: 19 - DNA, 16 bp, Artificial
  Synthetic oligonucleotide
gggggggggggagcct

SEQ ID NO: 20 - DNA, 18 bp, Artificial
  Synthetic oligonucleotide
tggggggggggggggggt

SEQ ID NO: 21 - DNA, 6 bp, Artificial
  Synthetic oligonucleotide
gagcct

SEQ ID NO: 22 - DNA, 20 bp, Artificial
  Synthetic oligonucleotide
aaaaaaaaaaaaaaaaaaaa

SEQ ID NO: 23 - DNA, 6 bp, Artificial
  Synthetic oligonucleotide
cccggg

SEQ ID NO: 24 - DNA, 18 bp, Artificial
  Synthetic oligonucleotide
ttgggggggggggggggg

SEQ ID NO: 25 - DNA, 20 bp, Artificial
  Synthetic oligonucleotide
cgcgcgcgcgcgcgcgcgcg

SEQ ID NO: 26 - DNA, 20 bp, Artificial
  Synthetic oligonucleotide
cgcgcgcgcgcgcgcgcgcg

SEQ ID NO: 27 - DNA, 16 bp, Artificial
  Synthetic oligonucleotide
gggggggggggacgct

SEQ ID NO: 28 - DNA, 18 bp, Artificial
  Synthetic oligonucleotide
ttttttttttttttttgg

SEQ ID NO: 29 - DNA, 12 bp, Artificial
  Synthetic oligonucleotide
cgcgcgcgcgcg

SEQ ID NO: 30 - DNA, 12 bp, Artificial
  Synthetic oligonucleotide
cgcgcgcgcgcg

SEQ ID NO: 31 - DNA, 20 bp, Artificial
  Synthetic oligonucleotide
ggggggggggaaaaaaaaaa

SEQ ID NO: 32 - DNA, 16 bp, Artificial
  Synthetic oligonucleotide
ggggggggggcccgga

SEQ ID NO: 33 - DNA, 16 bp, Artificial
  Synthetic oligonucleotide
cgcgcgcgcgcgcgcg

SEQ ID NO: 34 - DNA, 9 bp, Artificial
  Synthetic oligonucleotide
ggggggggg

SEQ ID NO: 35 - DNA, 6 bp, Artificial
  Synthetic oligonucleotide
tttttt

SEQ ID NO: 36 - DNA, 17 bp, Artificial
  Synthetic oligonucleotide
ggggggggggggggggg

SEQ ID NO: 37 - DNA, 5 bp, Artificial
  Synthetic oligonucleotide
ggggg

SEQ ID NO: 38 - DNA, 17 bp, Artificial
  Synthetic oligonucleotide
tgggggggggggggggg

SEQ ID NO: 39 - DNA, 8 bp, Artificial
  Synthetic oligonucleotide
cgcgcgcg

SEQ ID NO: 40 - DNA, 11 bp, Artificial
  Synthetic oligonucleotide
ttttttttttt

SEQ ID NO: 41 - DNA, 14 bp, Artificial
  Synthetic oligonucleotide
gggggggggggggg

SEQ ID NO: 42 - DNA, 17 bp, Artificial
  Synthetic oligonucleotide
gtttttttttttttttt

SEQ ID NO: 43 - DNA, 20 bp, Artificial
  Synthetic oligonucleotide
gggggggggggggggggggg

SEQ ID NO: 44 - DNA, 20 bp, Artificial
  Synthetic oligonucleotide
gagagagagagagagagaga

SEQ ID NO: 45 - DNA, 20 bp, Artificial
  Synthetic oligonucleotide
gtgtgtgtgtgtgtgtgtgt

SEQ ID NO: 46 - DNA, 4 bp, Artificial
  Synthetic oligonucleotide
gggg

SEQ ID NO: 47 - DNA, 11 bp, Artificial
  Synthetic oligonucleotide
ggggggggggg

SEQ ID NO: 48 - DNA, 4 bp, Artificial
  Synthetic oligonucleotide
tttt

SEQ ID NO: 49 - DNA, 5 bp, artificial
  Synthetic oligonucleotide
ttttt

SEQ ID NO: 50 - DNA, 9 bp, Artificial
  Synthetic oligonucleotide
ttttttttt

SEQ ID NO: 51 - DNA, 4 bp, Artificial
  Synthetic oligonucleotide
cgcg

SEQ ID NO: 52 - DNA, 6 bp, Artificial
  Synthetic oligonucleotide
cgcgcg

SEQ ID NO: 53 - DNA, 8 bp, Artificial
  Synthetic oligonucleotide
cgcgcgcg

SEQ ID NO: 54 - DNA, 10 bp, Artificial
  Synthetic oligonucleotide
cgcgcgcgcg

SEQ ID NO: 55 - DNA, 16 bp, Artificial
  Synthetic oligonucleotide
cgcgcgcgcgcgcgcg

SEQ ID NO: 56 - DNA, 4 bp, Artificial
  Synthetic oligonucleotide
cgcg

SEQ ID NO: 57 - DNA, 6 bp, Artificial
  Synthetic oligonucleotide
cgcgcg

SEQ ID NO: 58 - DNA, 10 bp, Artificial
  Synthetic oligonucleotide
cgcgcgcgcg

SEQ ID NO: 59 - DNA, 6 bp, Artificial
  Synthetic oligonucleotide
gacgct

SEQ ID NO: 60 - DNA, 16 bp, Artificial
  Synthetic oligonucleotide
gggggggggggacgtt

SEQ ID NO: 61 - DNA, 6 bp, Artificial
  Synthetic oligonucleotide
gagctt

SEQ ID NO: 62 - DNA, 16 bp, Artificial
  Synthetic oligonucleotide
gggggggggggagctt

SEQ ID NO: 63 - DNA, 6 bp, Artificial
  Synthetic oligonucleotide
cccgga

SEQ ID NO: 64 - DNA, 22 bp, Artificial
  Synthetic oligonucleotide
tccataacgttgcaacgttctg

SEQ ID NO: 65 - DNA, 30 bp, Artificial
  Synthetic oligonucleotide
gggggggggggggggggggggggggggggg

SEQ ID NO: 66 - DNA, 40 bp, Artificial
  Synthetic oligonucleotide
gggggggggggggggggggggggggggggggggggggggg

SEQ ID NO: 67 - DNA, 20 bp, Artificial
  Synthetic oligonucleotide
gcgcgcgcgcgcgcgcgcgc